SEQUENCE LISTING (94 sequences)

SEQ ID NO: 1 - DNA, 705 bp, Homo sapiens
  CDS (21)..(704)
gaattcgcggccgcgtcgaccgtgaccccaccatgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcagcaggagtcacagtcacagtcgcagtcacctgtggggccgcagggcagcagcagctcagcctctgggcctggggcttnccctggtggatctgangcaggcagccagggctccgggggaaggcgagggtgtccagctaacagcggctcaa

SEQ ID NO: 2 - protein, 228 aa, Homo sapiens
  misc_feature (205)..(205): The ′Xaa′ at location 205 stands for Tyr, Cys, Ser, or Phe.
RDPTMSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQQESQSQSQSPVGPQGSSSSASGPGAXPGGSXAGSQGSGGRRGCPANSGS

SEQ ID NO: 3 - DNA, 386 bp, Homo sapiens
  CDS (36)..(386)
gaattcgcggccgcgtcgacgtcgacgcggccgcggtcccagcgaacccgcgtgcaacctgtcccgactctagccgcctcttcagctcgccatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatgctgggacagccccgctcccagatgtaaagaacgcgacttccacaaacctggattttttatgtacaaccctgaccgtgaccgtttgctatattcctttttctatgaaa

SEQ ID NO: 4 - protein, 117 aa, Homo sapiens
VPANPRATCPDSSRLFSSPWIPTAPAPPVTPAPAPAPANAKSANAPPARKAAAPAALWAVPSVPRAASAKGRRTSAAAAPDAGTAPLPDVKNATSTNLDFLCTTLTVTVCYIPFSMK

SEQ ID NO: 5 - DNA, 443 bp, Homo sapiens
  misc_feature (13)..(413): n represents a, c, t or g
tggaacagtgganganagctttgtatatntatntatnnatntatntatntatntatntatntaaactnnataggagnacntagaggagtgcctaatanatntanngcncgannccanatacntgntanatnngagncagcncttttntgggggncatctattaacaagagtgtaaantanattannnacntgtnntataatcagcanagattnatcnntnacacgcaaanngcncttangggagtanncanacaactaagtgccnctanctaantggcntgngaaaacaganncacnctgnacataatattancagagaggggngatatatgngatatgcngggnggaaantgngacncttcnattcacaacagtgntgccccaantggaaccatnatagggggnnggnnggnactaacatctctagtcagcgagctggcaga

SEQ ID NO: 6 - DNA, 106 bp, Homo sapiens
  CDS (24)..(104)
gaattcgcggccgcgtcgacgggggttgcagtgagccgagatcgtgccactgtactccagcctgggcaacagagcaagactccatctcagccaaaaataataataa

SEQ ID NO: 7 - protein, 27 aa, Homo sapiens
GCSEPRSCHCTPAWATEQDSISAKNNN

SEQ ID NO: 8 - DNA, 401 bp, Homo sapiens
  CDS (126)..(401)
gaattcgcggccgcgtcgacgatcagggttcacagaggagggaactgaatgacatcccaggattacataaactgtcagaggcagccgaagagttcacaagtgtgaagcctggaagccggcgggtgccgctgtgtaggaaagaagctaaagcacttccagagcctgtccggagctcagaggttcggaagacttatcgaccatggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtcccgaagccttccagtgtgtgtctgcagtaagaaggtgggagaatccatgccggcggtcttcaacc

SEQ ID NO: 9 - protein, 92 aa, Homo sapiens
PLCRKEAKALPEPVRSSEVRKTYRPWSARPACCCCCCRWCTSLRPRQNLVSWTMKISAASATSPNLSPTGPEAFQCVSAVRRWENPCRRSST

SEQ ID NO: 10 - DNA, 710 bp, Homo sapiens
  CDS (30)..(710)
gaattcgcggccgcgtcgacctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaggatcagcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctg

SEQ ID NO: 11 - protein, 227 aa, Homo sapiens
NKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPEL

SEQ ID NO: 12 - DNA, 320 bp, Homo sapiens
  CDS (21)..(320)
gaattcgcggccgcgtcgacacgagatcttccttccgctctgaggcaaccccctcggagcctgtgttagtgtctgtccatctgtctgtcctacccgcccgcgtccaaccccggggcatggacagggccagggttgcggtcgcggctgggagcctcgcccctccagtgttgcctcctcccatccagcgtctgcgcggatgtagcatgttctatgtgtttttaaacgaagatccgagcgacggctcctccccgatccccgacagtggctctccaagcggcccccgggcagccccagagcaccccgcccgactcccattaacc

SEQ ID NO: 13 - protein, 100 aa, Homo sapiens
TRSSFRSEATPSEPVLVSVHLSVLPARVQPRGMDRARVAVAAGSLAPPVLPPPIQRLRGCSMFYVFLNEDPSDGSSPIPDSGSPSGPRAAPEHPARLPLT

SEQ ID NO: 14 - DNA, 281 bp, Homo sapiens
  CDS (21)..(281)
gaattcgcggccgcgtcgacaaaaggataaatctcatttcacagacaaagagaccggacaggaacatgagcttatcgagagcatgcccctgttggaatggtttgctaacaactataaaaaatttggagctactgttggaaattgtcacagataaatcacaagaagggtctcagtttgtgaaaggatttggtggaattggaggtatcttgcggtaccgagtagatttccagggaatggaataccaaggaggagacgatgaattttttgaccttgatgactac

SEQ ID NO: 15 - protein, 87 aa, Homo sapiens
KRINLISQTKRPDRNMSLSRACPCWNGLLTTIKNLELLLEIVTDKSQEGSQFVKGFGGIGGILRYRVDFQGMEYQGGDDEFFDLDDY

SEQ ID NO: 16 - DNA, 170 bp, Homo sapiens
  CDS (21)..(170)
gaattcgcggccgcgtcgacccgctgcccccgccatgtctgctaccgctgccacggccccccctgctgccccggctggggagggtggtccccctgcaccccctccaaacctcaccagtaacaggagactgcagcagacccaggcccaggtggatgaggtggtggacatca

SEQ ID NO: 17 - protein, 50 aa, Homo sapiens
PLPPPCLLPLPRPPLLPRLGRVVPLHPLQTSPVTGDCSRPRPRWMRWWTS

SEQ ID NO: 18 - DNA, 554 bp, Homo sapiens
  CDS (381)..(554)
naattcgcggccgcgtcgacgactccggctatgtcatcgccctgcgcagcgtacatcactgacaacatgcagnctcctcagcttccaccgtggggacctcatcaagctgctgccggnggccaccctggagccaggctggcagtttggctctgccgggggccgttccggactctttcctgccgacatagtgcagccggctgccgctcccgacttttccttctccaaggagcagaggagtggctggcacaagggtcagctgtccaacggggaaccagggctggctcggtgggacagggcctcagagcgccctgcccacccttggagccaggcacacagtgacgactcggaggccaccagcctgtcctctgtggcctatgcctttctgcccgactcccacagctacaccatgcaggaattcgcccggcgttacttccggaggtcccaggccttgctgggccagactgatggaggtgccgcaggaaaggacacggacagcctggtgcagtacaccaaggctcccatccaggagtcgctcctcagcctcagtgatgatg

SEQ ID NO: 19 - protein, 58 aa, Homo sapiens
FCPTPTATPCRNSPGVTSGGPRPCWARLMEVPQERTRTAWCSTPRLPSRSRSSASVMM

SEQ ID NO: 20 - DNA, 388 bp, Homo sapiens
  CDS (21)..(386)
gaattcgcggccgcgtcgacaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcnccttgctgaagcccaacatggtcaccccaagccatgctttcactca

SEQ ID NO: 21 - protein, 122 aa, Homo sapiens
  misc_feature (108)..(108): The ′Xaa′ at location 108 stands for Thr, Ala, Pro, or Ser.
NGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGXLLKPNMVTPSHAFT

SEQ ID NO: 22 - DNA, 284 bp, Homo sapiens
  CDS (21)..(284)
gaattcgcggccgcgtcgaccaaggagaaggcaaaggagacggcactggcagctacagagaaggccaaggacctcgccagcaaggcggccaccaagaagcagcagcagcagcaacagtttgtgtagccagtctaccaccaccacagcaccccagacagctaggcttagcccctctgccctcccttcattgtactttatcattaaaaatcaacttccagccctgtctgctgtctacgtggtgggttgtggggatgcagtttggcatctgcagtacaccaagcaca

SEQ ID NO: 23 - protein, 88 aa, Homo sapiens
QGEGKGDGTGSYREGQGPRQQGGHQEAAAAATVCVASLPPPQHPRQLGLAPLPSLHCTLSLKINFQPCLLSTWWVVGMQFGICSTPST

SEQ ID NO: 24 - DNA, 323 bp, Homo sapiens
  CDS (45)..(323)
gaattcgcggccgcgtcgacgaaagaggtcaccaagggcagaggtgtccaggccggagccaggggccccactgttgggatgctggctgcagtggggcgccccaagcccaggtcccctctgtcttctctttcgactttgcagctgtacttgttttgctcctctacccgcaggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagacccccca

SEQ ID NO: 25 - protein, 93 aa, Homo sapiens
CPGRSQGPHCWDAGCSGAPQAQVPSVFSFDFAAVLVLLLYPQELTWTQILGPPWSASSSAFGSGKNSSRTFYSQRQSLSFLCPICISSRRDPP

SEQ ID NO: 26 - DNA, 400 bp, Homo sapiens
  CDS (21)..(398)
gaattcgcggccgcgtcgacgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcgggttctcatgctgcaacaagctggccatgcacgaagttcatgatcctcccagtcggtgcagcaaacttcagggaagcctgcgcattggaacagaggg

SEQ ID NO: 27 - protein, 126 aa, Homo sapiens
VSKAVEHINKTIAPALVSKKLNVTEQEKIDKLMIEMDGTENKSKFGANAILGVSLAVCKAGAVEKGVPLYRHIADLAGNSEVILPVPAFNVINGGFSCCNKLAMHEVHDPPSRCSKLQGSLRIGTE

SEQ ID NO: 28 - DNA, 683 bp, Homo sapiens
  CDS (21)..(683)
gaattcgcggccgcgtcgacgacgaagacacagagggctttggagtccctcgccacgaccctttgcctgaccccctcaaggtccccccaccactgcctgacccatccagcatctgcatggtggaccccgagatgctgccccccaagacagcacggcaaacggagaacgtcagccgcacccggaagcccctggcccgccccaactcacgcgctgccgcccccaaagccactccagtggctgctgccaaaaccaaggggcttgctggtggggaccgtgccagccgaccactcagtgcccggagtgagcccagtgagaagggaggccgggcacccctgtccagaaagtcctcaacccccaagactgccactcgaggcccgtcggggtcagccagcagccggcccggggtgtcagccaccccacccaagtccccggtctacctggacctggcctacctgcccagcgggagcagcgcccacctggtggatgaggagttcttccagcgcgtgcgcgcgctctgctacgtcatcagtggccaggaccagcgcaaggaggaaggcatgcgggccgtcctggacgcgctactggccagcaagcagcattgggaccgtgacctgcaggtgaccctgatccccactttcgactcggtggccatgcatacgtggtacgcagagacgcacgcccgg

SEQ ID NO: 29 - protein, 221 aa, Homo sapiens
DEDTEGFGVPRHDPLPDPLKVPPPLPDPSSICMVDPEMLPPKTARQTENVSRTRKPLARPNSRAAAPKATPVAAAKTKGLAGGDRASRPLSARSEPSEKGGRAPLSRKSSTPKTATRGPSGSASSRPGVSATPPKSPVYLDLAYLPSGSSAHLVDEEFFQRVRALCYVISGQDQRKEEGMRAVLDALLASKQHWDRDLQVTLIPTFDSVAMHTWYAETHAR

SEQ ID NO: 30 - DNA, 611 bp, Homo sapiens
  CDS (21)..(611)
gaattcgcggccgcgtcgacgacagaagattttcgcgttctgcgataccagagcagataatcagctcaacactgtcctcaccatcaagtaatgccccagacccatgtgcaaaggagactgtactgagtgccctcaaagagaagaagaagaaaaggacagtggaggaagaagaccaaatattccttgatggccaggaaaataaaagaagctgtcttgtcgacggtctcactgatgcctcttctgcattcaaagttcctcgacccgggccagatacactccagttcacagtggatgtcttccactttgctaatgactccagaaacatgatatacatcacctgccacctgaaggtcaccctagctgagcaggacccagatgaactcaacaaggcctgttccttcagcaagccttccaacagctggttcccagtggaaggcccggctgacatctgtcaatgctgtaacaaaggtgactgtggcactccaagccattccaggaggcagcctcgtgtcgtgagccagtggtccacgtctgcttcccgtaaccgcaggcatgtgacagaagaagcagatgtcaccgtgggggccactgatcttcctggacaggagtgg

SEQ ID NO: 31 - protein, 197 aa, Homo sapiens
DRRFSRSAIPEQIISSTLSSPSSNAPDPCAKETVLSALKEKKKKRTVEEEDQIFLDGQENKRSCLVDGLTDASSAFKVPRPGPDTLQFTVDVFHFANDSRNMIYITCHLKVTLAEQDPDELNKACSFSKPSNSWFPVEGPADICQCCNKGDCGTPSHSRRQPRVVSQWSTSASRNRRHVTEEADVTVGATDLPGQEW

SEQ ID NO: 32 - DNA, 425 bp, Homo sapiens
  CDS (18)..(425)
gaattcgcggccgcgtcgacggcgagccctgcttctcatgcctctacggcaacggcccgcacccgccggccccgcagccctccagcaggttcaacgacgcgcccgcggccgacaaggagcccagcgtggtgcagttctgtgagttcgtatctgccccagaggtgagccgctgggcggggcccatcatcgatgtcctcctggactacgtgggcaacgtgcagctctgctcgcggctgaaggaacacatcgacagctttgaggactgggccgtcatcaaggagaaggcagaacctccaagacctctggctcacctttgccgactgcgggttcgaaaggccattgggaaataccgtataaaactcctagacaccttgccgctcccaggcaggctgattagatacctgaaatacgagaacacccagtaa

SEQ ID NO: 33 - protein, 135 aa, Homo sapiens
DGEPCFSCLYGNGPHPPAPQPSSRFNDAPAADKEPSVVQFCEFVSAPEVSRWAGPIIDVLLDYVGNVQLCSRLKEHIDSFEDWAVIKEKAEPPRPLAHLCRLRVRKAIGKYRIKLLDTLPLPGRLIRYLKYENTQ

SEQ ID NO: 34 - DNA, 206 bp, Homo sapiens
  CDS (102)..(206)
gaattcgcggccgcgtcgaccaccgntccgtggtccccgggngcgcattnanancgctnccgncntccannngtcactgantgncccggnccnctgcgnccggaacctgncccaactcctanagctggtccttggggagacgcgggtcccatganctgcggctactactggggagtgcgctggtctgtaagattgttggactctcg

SEQ ID NO: 35 - protein, 35 aa, Homo sapiens
  misc_feature (3)..(3): The ′Xaa′ at location 3 stands for Trp, or Cys.
GTXPNSXSWSLGRRGSHXLRLLLGSALVCKIVGLS

SEQ ID NO: 36 - DNA, 302 bp, Homo sapiens
  CDS (21)..(302)
gaattcgcggccgcgtcgaccttgaacagaagctggctttggcagaacggattcgaggccacgtcctgtcattggcactacagatgtatggctgccgtgttatccagaaagctcttgagtttattccttcagaccagcagaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatcct

SEQ ID NO: 37 - protein, 94 aa, Homo sapiens
LEQKLALAERIRGHVLSLALQMYGCRVIQKALEFIPSDQQNEMVRELDGHVLKCVKDQNGNHVVQKCIECVQPQSLQFIIDAFKGQVFALSTHP

SEQ ID NO: 38 - DNA, 143 bp, Homo sapiens
  CDS (21)..(143)
gaattcgcggccgcgtcgacgcagttcgggatcatttgaagaaagatagtcagaacaagactttacttaaaaccctggcagaattgtacacctatgacaagaactatggcaatgctctggaaatatacnttaacattaagaca

SEQ ID NO: 39 - protein, 41 aa, Homo sapiens
  misc_feature (37)..(37): The ′Xaa′ at location 37 stands for Ile, Val, Leu, or Phe.
AVRDHLKKDSQNKTLLKTLAELYTYDKNYGNALEIYXNIKT

SEQ ID NO: 40 - DNA, 293 bp, Homo sapiens
  CDS (21)..(293)
gaattcgcggccgcgtcgacccagattctaatgccataaggccccagctggttccccatcacagaaccacccagcaggaagccgggggtacaatgacctcggccacacagagcgctcgtgctttcagcagagcgcctccacagacaagcctgtggccagccctgccctccagcctcgagctggagttgctccagaggcagccccagcaggcctccctccacatagaggtggctgggccaattgcccaaacacgtgagacacacagggagctttatgtgttcatcttcctgctt

SEQ ID NO: 41 - protein, 91 aa, Homo sapiens
PDSNAIRPQLVPHHRTTQQEAGGTMTSATQSARAFSRAPPQTSLWPALPSSLELELLQRQPQQASLHIEVAGPIAQTRETHRELYVFIFLL

SEQ ID NO: 42 - DNA, 708 bp, Homo sapiens
gaattcgcggccgcgtcgaccatctctgctgagaaagcctaccatgaacagctttctgtagcagagatcaccaatgcttgctttgagccagccaaccagatggtgaaatgtgaccctcgccatggtaaatacatggcttgctgcctgttgtaccgtggtgacgtggttcccaaagatgtcaatgctgccattgccaccatcaagaccaagcgtaccatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaaacgtgcctttgttcactggtacgttggggaggggatggaggaaggtgagttttcagaggcccgtgaggacatggctgcccttgagaaggattatgaggaggttggtgtggattctgttgaaggagagggtgaggaagaaggagaggaatactaaagttaaaacgtcacaaaggtgctgcttttacagggaagcttattctgttttaaacattgaaaagttgtggtctgatcagttaatttgtatgtagcagtgtatgctctcatatacaattactgacctatgctctaaacatgaatgctttgttacaga

SEQ ID NO: 43 - DNA, 83 bp, Homo sapiens
  CDS (21)..(83)
gaattcgcggccgcgtcgacggctgcttaagttttgtatgtagttggattttttggagtccgaaggtatccatctgcagaaat

SEQ ID NO: 44 - protein, 21 aa, Homo sapiens
GCLSFVCSWIFWSPKVSICRN

SEQ ID NO: 45 - DNA, 140 bp, Homo sapiens
  CDS (21)..(140)
naattcgcggccgcgtcgacggactcctgagcagagtgtcgagatgtccacagaaggaggatttggtggtactagcagcagtgatgcccagcaaagtctacagtcgttntggcctcgggtcatggaagaaatccggaatt

SEQ ID NO: 46 - protein, 40 aa, Homo sapiens
  misc_feature (30)..(30): The ′Xaa′ at location 30 stands for Tyr, Cys, Ser, or Phe.
GLLSRVSRCPQKEDLVVLAAVMPSKVYSRXGLGSWKKSGI

SEQ ID NO: 47 - DNA, 709 bp, Homo sapiens
  misc_feature (1)..(701): n represents a, c, t or g
naattcgcggccgcgtcgacccaggtcttcagccccacgtcccctccctgaacaatcccttctcatgaanttcagcagtcaanaaatctgcagctggaagtaaatgaataaatgttttgttttcanaaataaaaaaaaaanacccgacanaancnanttnttatgnctactttgaaanaacnccnngggangacanaannctgcnaattaaggaaggtgaactatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgntagaaacttctggtaacttanatggtctggaatacaagttacatgattttggctacagaggagtctcttcccaagagactgctggcataggagcatctgctcacttggntaacttca

SEQ ID NO: 48 - DNA, 447 bp, Homo sapiens
  misc_feature (406)..(406): n represents a, c, t or g
gaattcgcggccgcgtcgactgctgagcgaggtgatacaactgatgcagtatgtcatctatggaattgcgtcctttttcttcttgtatgggatcattctgttggagaaggcttttacaccacaagtgcagtgaaagaactgcacggtgagtttaaaacaaccgcttgtggccgatgcatcagtggaatgttcgttttcctcacctatgtgcttggagtggcctggctgggtgtgtttggtttctcagcggtgcccgtgtttatgttctacaacatatggtcaacttgtgaagtcatcaagtcaccgcagaccaacgggaccacgggtgtggagcagatctgtgtggatatccgacaatacggtatcattcctggaatgctttcccggaaaaatatgtggctcttgncctgggagaacatctgcaacacaaacgaagttctacatgtc

SEQ ID NO: 49 - DNA, 590 bp, Homo sapiens
  CDS (21)..(590)
gaattcgcggccgcgtcgaccagagcctgggcctgcttgaccagaacccccacctggctcaggagctgcggccccaaatccaggaactcctccactctgaacacctgggtcccagtgaattggaagcccctgcccctgggggcagcagcgaggacaagggtgggctgcagcctccagattccaaggatgcagacacccccatgacccttccaaaagggtccacagaacaagatgctgcatcccctgagaaagagaagatgaacccgctggaacagtatgagcgaaaggtgaatgcgtctgttccaaggggtttccctttccactcatcggagattcagagggatgagctggcaccagctgggacaggggtgtcccgtgaggctgtgtcgggtctgctgatcatgggagcgggcggaggctccctcatcgtcctctccatgctgctcctgcgcaggaagaagccctacggggctatcagccatggcgtggtggaggtggaccccatgctgaccctggaggagcagcagctccgcgaactgcagcggcacggctatgagaaccccacttaccgcttcctggaggaacgaccc

SEQ ID NO: 50 - protein, 190 aa, Homo sapiens
QSLGLLDQNPHLAQELRPQIQELLHSEHLGPSELEAPAPGGSSEDKGGLQPPDSKDADTPMTLPKGSTEQDAASPEKEKMNPLEQYERKVNASVPRGFPFHSSEIQRDELAPAGTGVSREAVSGLLIMGAGGGSLIVLSMLLLRRKKPYGAISHGVVEVDPMLTLEEQQLRELQRHGYENPTYRFLEERP

SEQ ID NO: 51 - DNA, 446 bp, Homo sapiens
  CDS (117)..(446)
gaattcgcggccgcgtcgaccatgaacgaaagagaagcaaaagtaaggaacggaagcgaagtagagacagagaaaggaaaaagagcaaaagccgtgaaagaaagcgaagtagaagcaaagagaggcgacggagccgctcaagaagtcgagatcgaagatttagaggccgctacagaagtccttactccggaccaaaatttaacagtgccatccgaggaaagattgggttgcctcatagcatcaaattaagcagacgacgttcccgaagcaaaagtccattcagaaaagacaagagccctgtgagagaacctattgataatttaactcctgaggaaagagatgcaaggacagtcttctgtatgcagctggcggcaagaattcgaccaaggggatttggaagagtttttctttcagtaggaaaggntcgaggatgtgaggatgatttc

SEQ ID NO: 52 - protein, 110 aa, Homo sapiens
  misc_feature (103)..(103): The ′Xaa′ at location 103 stands for Asp, Gly, Ala, or Val.
KERRRSRSRSRDRRFRGRYRSPYSGPKFNSAIRGKIGLPHSIKLSRRRSRSKSPFRKDKSPVREPIDNLTPEERDARTVFCMQLAARIRPRGFGRVFLSVGKXRGCEDDF

SEQ ID NO: 53 - DNA, 496 bp, Homo sapiens
  misc_feature (1)..(490): n represents a, c, t or g
naattcgcggccgcgtcgacanaactttggagctggccttggctccacctgaccctgtgtcctgngggnccacncatgccggngctggggacctgtgtgcactttgnggggaacacctctatgtcctggaacgcctctgtgtcaacggncatttcttccaccggagctgcttccggtgccatacctgtgaggccacactgtggccaggnggctacgagcagcacccaggagatggacatttctactgcctccagcacctgccccagacagaccacaaagcggaaggcagcgatagaggccctgagagtccggtaaagactgaaggcgtgtatgtatattggngcatgggaagaggaaganggnctngtcaaaccancgctttnctgggggtaactgaatgtcntnttctactancatgtaanctctgngaangcnnggatgcctgnctgcttacacttnttgntctctnaantatntaaaacacanncanacactg

SEQ ID NO: 54 - DNA, 668 bp, Homo sapiens
  misc_feature (1)..(650): n represents a, c, t or g
naattcgcggccgcgtcgaccgtaaaagccccaatcttagcaagtgccggggaaaagcctgggtcgacctcaanctagttcttctggagctcaacttcttgccaaccacaaggggaccaagctgaccaaacagcagcctaattctggcccgngacatactggagatcggggcccaatggagcatcctacgcaaggacatcccctccttcgagcgctacatggcccagctcaaatgctactactttgattacaaggagcagctccccgagtcagcctatatgcaccagctcttgggcctcaacctcctcttcctgctgtcccagaaccgggtggctgagttccacacggagttggagcggctgcctgccaaggacatacagaccaatgtctacatcaagcacccagtgtccctggagcaatacctgatggagggcagctacaacaaagtgttcctggccaagggtaacatccccgccgagagctacaccttcttcattgacatcctgctcgacactatcagggatgagatcgctgggtgcatcgagaaggcctacgagaaaatccttttcactgaggccacccggatcctcttcttcaacacacccaaaaagatgacagactacgccaagaagcgangggtgggncctggnccccaacaactactacag

SEQ ID NO: 55 - DNA, 389 bp, Homo sapiens
gaattgcggccgcgtcgacatttggccggggcctttgtctctagctgctgcgggagctccaggtctagtctttactgctctgtgtattctgctcctagaggcccagcctctgtgactccgttatctgcaggtattgggagatgcacagctaagatgccaggaccacctggaagcctagaaatgggaccactgacatttagggatgtggccatagaattctctctggaggagtggcaatgcctggacactgcacaacggaatttatataggaaagtgatgtttgagaactacagaaacctggtcttcctgggtattgctgtctctaagcctcacctgataacctgtttggagcaaggaaaagagccctggaataggaagagacaggagat

SEQ ID NO: 56 - DNA, 239 bp, Homo sapiens
  CDS (21)..(239)
gaatcgcggccgcgtcgacgcagcagacacccatcccagatcctgtcgaggtgaggcctgggcgagtggatggacgtcacacagagtgtgcagcatgcttcctgggggaaggccatcgaggccaaagaccctcacagataaagcggaaggtcatgtctctgcacacagtctcacccccgcggctcagagtgcccatcgcgggctccttgggcagtgtttatctggggccacgcccgctc

SEQ ID NO: 57 - protein, 73 aa, Homo sapiens
QQTPIPDPVEVRPGRVDGRHTECAACFLGEGHRGQRPSQIKRKVMSLHTVSPPRLRVPIAGSLGSVYLGPRPL

SEQ ID NO: 58 - DNA, 323 bp, Homo sapiens
  CDS (21)..(323)
gaattcgcggccgcgtcgacggagacaggaagcctcaagttccagtgcagagatcctacttctctgagtcagctgaccccctccccgcaatccctcaaaccttgaggagaagtggggaccccacccctcatcaggagttccagtgctgcatgcgattatctacccacgtccacgcggccacctcaccctctccgcacacctctggctgtctttttgtactttttgttccagagctgcttctgtctggtttatttaggttttatccttccttttctttgagagttcgtgaagagggaagccaggattggggacctgatggagag

SEQ ID NO: 59 - protein, 101 aa, Homo sapiens
GDRKPQVPVQRSYFSESADPLPAIPQTLRRSGDPTPHQEFQCCMRLSTHVHAATSPSPHTSGCLFVLFVPELLLSGLFRFYPSFSLRVREEGSQDWGPDGE

SEQ ID NO: 60 - DNA, 711 bp, Homo sapiens
gaattcgcggccgcgtcgaccactgctccagttattatatttgtttccaaaatgtttgcagttgatgctaaggccttgcctcagaataagccaaggcctctcactcaagaagaaattgctcagagacgtgagcgtgcaagacaaaggcatgcagagaagcttgcagcagcacagggacaggcacccttggagcccacccaagatgggagtgccattgaaacatgtccaaaaggagaggagccaagaggtgacgagcaacaggtggaaagtatgacccctaaacctgtgctccaggaagaaaacaaccaagagtcttttattgcatttgctcgggtgttcagtggtgtggctcgaagaggaaagaaaatttttgtcttggggcccaaatacagtcctcttgagtttttacgaagggtaccattaggcttctcagctccaccagatggcctcccccaagtcccccacatggcatactgtgctctggaaaacctgtatcttctgatgggaagggaactggaatatctagaggaggtacctccaggaaatgtgctaggaataggaggccttcaagattttgtgctgaaatctgcaacactgtgtagcctgccatcctgcccaccatttataccactcaacttcgaagccactcctattgtgagagttgctgttgaaccaaaacatccaagtgaaatgcctcagctcgtaaaagga

SEQ ID NO: 61 - DNA, 332 bp, Homo sapiens
gaattcgcggccgcgtcgaccattcagtcttctagcatttggagctaggtaatgatatttttttttctcaccagtcatatttaccatggtagtaaaagtcctagccagtacagttaggcaaaaataaatgaataaaatgcatatatatccgggaagagagaaattaaacttcctctattcataggagatagattctttcatgtagaaaagtgtaaagaaattataaaaaaaactcctcggaccaatcagtgagtttagcaaggttgcagggtacaaagttaatatataaaaatcgattatatgccagcagtgaacaattggagtttgaaatt

SEQ ID NO: 62 - DNA, 709 bp, Homo sapiens
  CDS (105)..(707)
gaattcgcggccgcgtcgacctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagct

SEQ ID NO: 63 - protein, 201 aa, Homo sapiens
MQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQ

SEQ ID NO: 64 - DNA, 122 bp, Homo sapiens
  CDS (21)..(122)
gaattcgcggccgcgtcgacaatatgaagaacaggatgatgtggagagtaatgactctggagaagaccagagtgaatataatacagacacatctgctctgtcttctattaatccagatgaaa

SEQ ID NO: 65 - protein, 34 aa, Homo sapiens
NMKNRMMWRVMTLEKTRVNIIQTHLLCLLLIQMK

SEQ ID NO: 66 - DNA, 209 bp, Homo sapiens
  CDS (21)..(209)
gaattcgcggccgcgtcgacaggagggacccggagttgcccgtcacgtccttcgactgcgccgtgtgccttgaggtgttacaccagcctgtccggacccgctgtggccacngtattctgccgttcctgtattgctaccagtctaaagaacaacaagtggacctgtccttattgccgggcatatcttccttcagaaggagttccagcaac

SEQ ID NO: 67 - protein, 63 aa, Homo sapiens
  misc_feature (31)..(31): The ′Xaa′ at location 31 stands for Ser, Gly, Arg, or Cys.
RRDPELPVTSFDCAVCLEVLHQPVRTRCGHXILPFLYCYQSKEQQVDLSLLPGISSFRRSSSN

SEQ ID NO: 68 - DNA, 709 bp, Homo sapiens
  misc_feature (705)..(705): n represents a, c t or g
gaattcgcggccgcgtcgaccttttggtttatatatattttttttaatacctcagtgctgcaagtatcaccagagaggctatggaagaatttttttttaatttattgtagatgtaaacagaattttaaaaataaaaagtataaacatcactgcacttgtgactggtgggaaaaactgacagtttcctctttgcacatgtttaacatttggctgttataatatatggtcctcggttggggaaagatacttatgatgaaggatattttttaatttaacttttttttaaatattggtaataggtcggcaacagcaactatagaagtacaactcaatagatggcattaaaacatattgtagtgtggatatatattttttcttttttaaaatgtgatattgacgttttattaatattttttaaattgttacgtttataaatttggtacttaaggcacagccagtatgagacactgaatgcgacatttattataaagagctgctgcactcctatttttataaattttactaacaaagtagactaatgtagacattcacagacatggtagggcaaaagcatcttcaaactaaagactccaaaatgctaactcagaaagaaagaaaaaacccgttttcaattctaatgaaacagcaccaccattttttttaattaaaaaaaaaaacatggtctttggttttctaaaaaaaggnaggg

SEQ ID NO: 69 - DNA, 706 bp, Homo sapiens
gaattcgcggccgcgtcgacctgagctgaactgcagctgggtgcacctggacctgggcagtgatgtggcccagcccctcctgactctgcctggcacagcctgtggccctggcctggtgtgtatagaccatcgatgccagcgtgtggatctcctgggggcacaggaatgtcgaagcaaatgccatggacatggggtctgtgacagcaacaggcactgctactgtgaggagggctgggcaccccctgactgcaccactcagctcaaagcaaccagctccctgaccacagggctgctcctcagcctcctggtcttattggtcctggtgatgcttggtgccagctactggtaccgtgcccgcctgcaccagcgactctgccagctcaagggacccacctgccagtacagtctcaggggccagccaagcccccacccccaaggaagccactgcctgccgacccccagggccggtgcccatcgggtgacctgcccggcccaggggctggaatcccgcccctagtggtaccctccagaccagcgccaccgcctccgacagtgtcctcgctctacctctgacctctccggaggttccgctgcctccaagccggacttagggcttcaagaggcgggcgtgccctctggagtcccctaccatgactgaaggcgccagagactggcggtgtcttaagactccgggcaccgccacgcg

SEQ ID NO: 70 - DNA, 116 bp, Homo sapiens
  CDS (21)..(116)
gaattcgcggccgcgtcgacgagaaatttgcacctaagaaacagggcgatggtggtaaggacctccccacggaagcaaagaagcgtgcagctgaattctgcagaggagctctatgc

SEQ ID NO: 71 - protein, 32 aa, Homo sapiens
EKFAPKKQGDGGKDLPTEAKKRAAEFCRGALC

SEQ ID NO: 72 - DNA, 254 bp, Homo sapiens
  CDS (21)..(254)
gaattcgcggccgcgtcgacgtggcttcaccgccggtcccttgcagcgctgcctttcgatctctccacatctcggtggcgcgggatctcaagatgcgcctccacctgctcctgctgctcgcgctgtgcggtgcaggcaccaccgccgcggagctcagttacagcttgcgtggcaactggagcatctgcaatgggaacggctcgctggagctgcccggggcggtccctggctgcgtgcacagcgccttgttccag

SEQ ID NO: 73 - protein, 78 aa, Homo sapiens
VASPPVPCSAAFRSLHISVARDLKMRLHLLLLLALCGAGTTAAELSYSLRGNWSICNGNGSLELPGAVPGCVHSALFQ

SEQ ID NO: 74 - DNA, 554 bp, Homo sapiens
  CDS (276)..(554)
gaattcgcggccgcgtcgacgagacattaaaatgtccagaggtagctggtgtgaaaaagttatatcgttgccttcagtgtaatagttttggaagcacttggtgagaaaacctaaagttgacgttttcttggggagaaacgatctccactattagggtgaaaaataaggagtcttctagaaaaggccttggggtgtgttatgtaagaaatactaagtcagttcactgtttccaggcttttcttacgaaagttaacagccacagctctttttggagcataaatgctgagcttttcctaaactctgaaacacttggcaacctctcagtcttgctaacagctattttttttttattttgttttcttttccaaatccctggcatactgctgaaatctggctgctgccgcccctggananacncccgtctgctgggggagtcaggctggggtacctgccagcgctcacgggctgngccactccaggcaggctcaacaactacttcaaaacanaaagcggctgtaattcacaattcagcccactacaggcaccgatacgtt

SEQ ID NO: 75 - protein, 93 aa, Homo sapiens
  misc_feature (46)..(46): The ′Xaa′ at location 46 stands for Lys, Arg, Thr, or Ile.
INAELFLNSETLGNLSVLLTAIFFLFCFLFQIPGILLKSGCCRPWXXXRLLGESGWGTCQRSRAXPLQAGSTTTSKTKAAVIHNSAHYRHRYV

SEQ ID NO: 76 - DNA, 218 bp, Homo sapiens
  CDS (36)..(218): n represents a, c, t or g
gaattcgcggccgcgtcgacgtcgacgcggccgggttaggcctccagtggcacgctgatggccgcctaactaaaaaactcaatggccttgctgatttagaggcttgcattaagacgcttcatggccaaggcttttctcagccaagtctaacaaccctgactgctttcagtgtggaggggtgcttgcaggagcattgtgtaattctaatccagagctgg

SEQ ID NO: 77 - protein, 61 aa, Homo sapiens
LGLQWHADGRLTKKLNGLADLEACIKTLHGQGFSQPSLTTLTAFSVEGCLQEHCVILIQSW

SEQ ID NO: 78 - DNA, 709 bp, Homo sapiens
  CDS (21)..(707)
gaattcgcggccgcgtcgacgggatgcagaccctcggccttcagcaccgctgccgaggtggctaccgggtcaaggccaggacgtcatatgtggatgagactctgtttggcagcccagcaggcacccggcctaccccaccggacttcgatccgccctgggtggagaaggctaacagaaccagaggcgtgggcaaggaggcatcgaaggccttgggggcaaaggggagctgtgagaccaccccctcaaggggcagcacccccaccctcacaccaaggaagaagaacaaatacagacccatcagccacaccccgtcttactgtgatgagtcgctgtttggctcccgatctgaaggcgccagcttcggggccccgcggatggcgaagggggatgccgcaaagctccgtgctctcttgtggacgccaccacctacccccaggggtagccactcgccccgccccagggaggcaccactgcgagccattcacccagctggtccctccaagacagagccggggccagcggcagactcccagaagttatctatgggtgggttacactcttcacgccccctgaagcggggactttcccattccctcacccacctgaatgtccccagcactggtcatccagccaccagtgccccccacacaaatgggcctcaggatctcangccttncacgtcaggggtgaccttncggagccccctggt

SEQ ID NO: 79 - protein, 229 aa, Homo sapiens
  misc_feature (217)..(217): The ′Xaa′ at location 217 stands for Lys, Arg, Thr, or Met.
GMQTLGLQHRCRGGYRVKARTSYVDETLFGSPAGTRPTPPDFDPPWVEKANRTRGVGKEASKALGAKGSCETTPSRGSTPTLTPRKKNKYRPISHTPSYCDESLFGSRSEGASFGAPRMAKGDAAKLRALLWTPPPTPRGSHSPRPREAPLRAIHPAGPSKTEPGPAADSQKLSMGGLHSSRPLKRGLSHSLTHLNVPSTGHPATSAPHTNGPQDLXPXTSGVTXRSPL

SEQ ID NO: 80 - DNA, 249 bp, Homo sapiens
gaattcgcggccgcgtcgaccgcggccgcgtcgacgagaggctgaggtgggaggattgcttgagaccagcctgggcaacatggtgaaactctgtctctacagaaaattttaaaaattagccaagtgtggtggtgcacacctgtagtcccagcagctggagaggctgaggcagaaggattgcttgagcccagaggcagaggtttcagtgagctgagactgcaccactggactccaacctaggtgacagag

SEQ ID NO: 81 - DNA, 122 bp, Homo sapiens
gaattcgcggccgcgtcgacccaggtcttcagccccacgtccctccctgaacaatcccttctcatgaaattcagcagtcaagaaatctgcagcggaagtaaatgaataaatgttttgttttc

SEQ ID NO: 82 - DNA, 613 bp, Homo sapiens
  misc_feature (495)..(602): n represents a, c t or g
gaattcgcggccgcgtcgacaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattatgttcttacaagcttaaagcttgatttgatctttgtttaaatgccaaaatgtacttaaatgagttacttagaatgccataaaattgcagtttcatgtatgtatataatcatgctcatgtatatttagttaccgtataatgctttctgagtgagttttactcttaaatcatttggttaaaatcatttggcttgctgtttactcccttctgtagtttttaattaaaaactttaagataagtctacattaacaatggatcacatctaaagctttatctttggggaatccaagttttttgggaaaaaccaaaatgggnnaattggcctaantggaattcaaggctttnaaagtcantattccgggctgggtaagggaatttatgagattacctgctctaaaaagtataaatggncnagggaccgntttggattgct

SEQ ID NO: 83 - DNA, 490 bp, Homo sapiens
  misc_feature (452)..(487): n represents a, c t or g
gaattcgcggccgcgtcgacgtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttaaaaaaaaaaaaaaaaaaaaaaaaaacccccnnnaaantttnnaatnggaaaannnnaaaanccccnggg

SEQ ID NO: 84 - DNA, 592 bp, Homo sapiens
  misc_feature (487)..(588): n represents a, c t or g
accccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaanaaaacacaaagngtataaatctgagtatatatttacatgtctttttaaaagggncgntaccananatttacccatcgnnnaanangctnctggnggntggnaggc

SEQ ID NO: 85 - DNA, 536 bp, Homo sapiens
catttgtgttttagtttcttttttcatgactgaagggagttttatgttattatttcttccatagttttgttttggtttatttttaacaacgctctagaaacaaagtcaaattaatcacaaaagacataattttgctttgttgtggaatttctatttcaacgtcaactctgtatctatgagtatgtctgttccacagacagatgaggcaggagtgatggggcactcaagaaagttcagaggaggcataagctatggaggttggaaaggaagaaagagaagagctgaagtaaatgtatggtagaaattaagacgtttctttgcaaaacaaggaaatcacacatatgcacacatacatatttatggttactatgattgttattatttgtccaatcaagcaaagcaccattatttctaacatataaaagaccagatcaaacacaatggaaataaagctactacatatatatgccagtatttctctagtaagattatgtttctcttactagaaaactattttctaaatattaacactgaaa

SEQ ID NO: 86 - DNA, 688 bp, Homo sapiens
  misc_feature (683)..(683): n reprsents a, c t or g
caaaaggtttgtgtccaggatgtacaaagaactctcaaaccggatagtaagaaaacaaacagcccaagtgaaaagcaggcaaaagacttgaatagacacttcaccaaagagcatacacgcgtggcaaacaagcacacgaaaagacgttcagccgccgatggcttggttataatttataacttacttatttttatctaataattgtagattcagtgtatttcttcaaaaaatgtttaattaaatgcatgttaatggtgagtgaatcccttgggtgacttcgtgtttaggtcgtattagggcatttgttggatcaacggatcattttaaccctgacttccccttattcccataaaagaagttttccagtggaatggagatttcattttgtcagcagcagtgaccacagccttaccaaagcagacgcgtgcgcgtgcacagatgcacacacacagatgtcttaaaagactagaatccacacttcctgagccagaggggccgtgttgacggtaatgcattctctatagagccaagtccaaactggcaagctcaatgatgcaggcaataaaccgcctttttggcagcctaccaatgccaaaaggataaatgtctttccaaaagtgtgtattcctgttaaattaagctcttgctaacttgaaaaatccctgttctgccagcgaagcttnctcct

SEQ ID NO: 87 - DNA, 402 bp, Homo sapiens
  misc_feature (115)..(183): n represents a, c, t or g
cgggccagggccacacccaagggcccagcgtgtcttgcctgcccgtgggcacccacggacgtggcttggtgctgaggatagcagagcccccagccatcactgctggcagcctggncaaaccgggtgagcaacaggaggacgaggggcctggggcggtgccaggctaccacaagaacctgcgtnttggaccattgcccctcccggccccaaaccacaggggctcaggtcgtgtgggccccagtgctagatctctccctcccttcgtctctgtctgtgctgttggcgacccctctgtctgtctccagccctgtctttctgttctcttatctctttgtttcaccttttccctctctggcgtccccggctgcttgtactcttggccttttctgtgtctcctttctg

SEQ ID NO: 88 - DNA, 568 bp, Homo sapiens
tatgaatggctccacgagggttcagctgtctcttacttttaaccagtgaaattgacctgcccgtgaagaggcgggcatgacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttag

SEQ ID NO: 89 - DNA, 30 bp, Homo sapiens
actaacatctctagtcagcgagctggcaga

SEQ ID NO: 90 - DNA, 30 bp, Unknown
  primer
ccggaattcgtcacccagtgccaaagctgt

SEQ ID NO: 91 - DNA, 49 bp, Unknown
  primer
cgggatcctctagagcggccgcttattatttagcttcctgtaacgcccc

SEQ ID NO: 92 - DNA, 2082 bp, Mus musculus
  CDS (1)..(2082)
gtcacccagtgccaaagctgtgtccaggcagctggagaggtaggggtactgaccggccactcccagaaatcacgtaggtcacccctggaggagaagcagttggaggaggaggattcctctgccacttccgaagaaggaggaggagggcctggcccagaagcttcactcaacaagggcctggccaagcacctgctgagtggtttgggggaccgactctgccgcctgctgcggaaggagcgggaggcccttgcctgggcacagcgagaaggccaggggccagccatgacagaggacagcccaggcattccacattgctgcagccgatgccaccacggactcttcaacacccactggagatgttcccactgtagccaccggctgtgtgtagcctgtggtcgcatagccggtgctggaaagaacagggagaaaacaggttctcaggaacagcacacagatgactgcgcccaggaggctgggcatgctgcctgttccctgatcctgacccagtttgtctccagccaggcgctggcagaactgagcactgtgatgcaccaagtctgggccaagtttgacattcgggggcactgtttctgccaggttgatgcccgtgtgtgggcccccggggatgggggtcagcagaaggaaccaacagagaaaactcccccaactccacaaccttcctgcaatggagattccaatcggaccaaggacatcaaagaagagaccccagactccactgagagcccagcagaggacggtgctggccggtcaccccttccttgtccctctctctgtgagctgctagcctctactgctgtcaaactctgcctggggcatgaccggattcacatggcctttgctccggtcaccccagctctgcccagtgatgaccgcattaccaacatcctggacagcattattgcgcaggtagtagaacggaagatccaagagaaagccctggggccaggcctgcgagcagggtcaggcttacgcaagggcctgagccttccattgtcaccagtgcgaacccggctgtctcctcctggagctttgctgtggctgcaggagcctaggcctaagcatggcttccatctcttccaggaacactggcggcagggccagcccgtgttagtgtcaggcatccagaagacattgagacttagcctgtggggaatggaagcccttgggacacttggtggccaggtgcagacactgactgcccttgggcctccccagcccacaaacctggacagcacagcattctgggagggattctctcatcctgagacacgtccaaagttagatgagggctctgtcctcctgctacaccgaaccctgggggataaggacgctagcagggtgcagaaccttgcctccagccttccactcccagaatactgtgcccaccaagggaaactcaacctagcgtcctacctccccctgggcctcacactgcatccactggagccccagctctgggcggcctatggtgtgaactcacaccgtggacacctggggaccaagaatctatgcgtggaggtgtctgacctaatcagtatcctggtgcacgccgaggcccagctgcctccctggtatcgagcacagaaagatttcctctcaggcctggatggggaaggactctggtctccagggagccagaccagcactgtgtggcatgtgttccgggcccaggatgcccagcgcatccgtcgctttctccagatggtgtgcccagctggagcaggaaccttggagcctggtgccccaggcagctgctacttggatgcagggttgcgccgacggctaagagaagagtggggtgtgagctgctggaccctgctgcaggctcctggggaagcggtgctggtcccggctggggcgccccatcaggtgcagggcctggtgagcacaatcagtgtcactcagcactttctgtctcctgagacctctgccctctctgctcagctctaccaccagggagccagcctaccccctgaccaccgtatgctttatgcccagatggaccgggctgtgttccaagcagtaaaggcggctgtgggggcgttacaggaagctaaatag

SEQ ID NO: 93 - protein, 693 aa, Mus musculus
VTQCQSCVQAAGEVGVLTGHSQKSRRSPLEEKQLEEEDSSATSEEGGGGPGPEASLNKGLAKHLLSGLGDRLCRLLRKEREALAWAQREGQGPAMTEDSPGIPHCCSRCHHGLFNTHWRCSHCSHRLCVACGRIAGAGKNREKTGSQEQHTDDCAQEAGHAACSLILTQFVSSQALAELSTVMHQVWAKFDIRGHCFCQVDARVWAPGDGGQQKEPTEKTPPTPQPSCNGDSNRTKDIKEETPDSTESPAEDGAGRSPLPCPSLCELLASTAVKLCLGHDRIHMAFAPVTPALPSDDRITNILDSIIAQVVERKIQEKALGPGLRAGSGLRKGLSLPLSPVRTRLSPPGALLWLQEPRPKHGFHLFQEHWRQGQPVLVSGIQKTLRLSLWGMEALGTLGGQVQTLTALGPPQPTNLDSTAFWEGFSHPETRPKLDEGSVLLLHRTLGDKDASRVQNLASSLPLPEYCAHQGKLNLASYLPLGLTLHPLEPQLWAAYGVNSHRGHLGTKNLCVEVSDLISILVHAEAQLPPWYRAQKDFLSGLDGEGLWSPGSQTSTVWHVFRAQDAQRIRRFLQMVCPAGAGTLEPGAPGSCYLDAGLRRRLREEWGVSCWTLLQAPGEAVLVPAGAPHQVQGLVSTISVTQHFLSPETSALSAQLYHQGASLPPDHRMLYAQMDRAVFQAVKAAVGALQEAK

SEQ ID NO: 94 - DNA, 33 bp, Unknown
  primer
ctattcgatgatgaagataccccaccaaaccca

(19) United States
(12) Patent Application Publication
(10) Pub. No.: US 2004/0086945 A1
(43) Pub. Date: May 6, 2004

(54) Hairless protein-interacting partner complexes and methods thereto

(75) Inventors: Kotikanyadanam Sreekrishna, Cincinnati, OH (US); Gina Sue Gerwe, Cincinnati, OH (US); Daniel Robert Toerner, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/452,858

(22) Filed: Jun. 2, 2003

Related U.S. Application Data
(60) Provisional application No. 60/385,414, filed on Jun. 3, 2002.

(51) Int. Cl. C12Q 1/68; G01N 33/53; C12N 5/06

(52) U.S. Cl. 435/7.1; 435/6; 435/354

(57) ABSTRACT

The present invention relates to complexes of hairless protein and proteins or nucleic acids identified as interacting with hairless protein using a yeast two-hybrid assay system. Hairless protein is indispensable for mammalian hair growth. Therefore, such interacting complexes are useful for screening test compounds for inhibition or enhancement of hair growth.

### CROSS REFERENCE TO RELATED APPLICATION

This application claims the benefit of U.S. Provisional Application No. 60/385,414, filed Jun. 3, 2002.

### FIELD OF INVENTION

The present invention relates to hairless protein-interacting partner complexes and methods therefor. The complexes are useful for screening test agents for inhibition or enhancement of hair growth, for example.

### BACKGROUND OF THE INVENTION

Removal or inhibition of unwanted hair can be cosmetically or medically desirable. Current methods for removing unwanted hair include use of hair removal creams, shaving, waxing and electrolysis. Such methods need to be repeated frequently or are time consuming and sometimes quite painful. Hair removal creams are commonly based on thioglycollates as the active ingredient such as NAIR, for example. Thioglycollates function as reducing agents at high pH by reducing disulfide bonds in hair. However, depilatory creams typically are not recommended for frequent use due to their high irritancy potential. Antiandrogens, which have been used to treat female hirsutism, may have unwanted side effects. Lasers treatments alone or in conjunction with topical formulations are generally not selective in that they result in only partial destruction of hair follicles, may be dependent upon the pigmentation of the hair, and may promote skin reaction.

Stimulation of desired hair growth also may be cosmetically or medically desirable. There are several forms of hereditary human hair loss known as alopecias. Alopecia universalis is caused by a rare inherited mutation in humans. Androgenic alopecia, or male pattern baldness, is largely the result of heredity, advancing age, and male hormone secretion, specifically the hormone dihydrotestosterone (DHT). Women are also affected by androgenic alopecia, especially those of Caucasian origin. Minoxidil has been used with some success by younger patients and patients with less hair loss. However, the degree of new hair growth is usually minimal, and the area of the scalp that is affected is usually limited. Finasteride is an orally administered inhibitor of 5 alpha-reductase, an enzyme that converts testosterone to its active metabolite DHT. Finasteride appears more effective than minoxidil in treating androgenic alopecia, however, side effects on libido, erection, and semen volume in men and fetal defects in pregnant women have been observed.

Hairless protein (HR) is an indispensable protein for mammalian hair growth and maintenance. Individuals with point mutations in HR are hairless but are in good health except for the total lack of hair. U.S. Pat. No. 6,348,348, Feb. 19, 2002, to Thompson, reports that Hr interacts directly and specifically with thyroid hormone receptor (TR) the same protein that regulates its expression. The '348 patent reports a comparison of the amino acid sequences of their human HR sequence, a human sequence published by Ahmad et al. (Science, 279, 720-724, 1998); a human sequence published by Cichon et al. (Hum. Mol. Genet., 7, 1671-1679 and erratum at 1987-1988, 1998); a rat sequence published by Thompson (J. Neurosci., 16, 7832-7840, 1996); and a mouse sequence published by Cachon-Gonzalez et al. (Proc. Natl. Acad. Sci. USA, 91, 7717-7721, 1994). WO 99/38965 reportedly provides an isolated nucleic acid that encodes human hairless protein.

Accordingly, there exists a need for selectively removing hair that is not time consuming, painful and damaging to the skin, and results in hair removal which is long lasting, and more permanent than conventional hair removal methods. There also exists a need to selectively stimulate or enhance hair growth that is equally consumer friendly. The present inventors provide herein protein and nucleic acid interacting partners of hairless protein that, together with HR, provide complexes useful for screening test agents for inhibition or enhancement of hair growth.

### SUMMARY OF THE INVENTION

The present invention provides compositions of hairless protein-hairless protein interacting partner complexes (HR-IP) determined by the present inventors using a yeast two-hybrid technology. The hairless protein interacting partners provided by the present invention are listed in Table 1 and include molecules involved in cell cycle, cell differentiation, transcription, protein turnover, protein processing, RNA splicing, house keeping (metallothionein, aldolase, enolase) and mitochondrial machinery (quinone oxidoreductase, cytochrome oxidase II and 16S RRNA, for example). Thus, Hr appears to be a well-connected, multi-functional protein. In particular, the interacting partners provided by the present invention interact with a truncated portion of hairless protein (Hrt) as set forth infra.

In one embodiment, a composition of the invention comprises a mouse Hrt protein-human interacting partner protein complex wherein the human interacting partner protein comprises a molecule selected from the group consisting of ubiquitous receptor UR, SEQ ID NO:4, MAPIA, SEQ ID NO:8, KIAA0930 protein, SEQ ID NO:13, and monocyte antigen CD14. In another embodiment, the human interacting partner protein is encoded by a nucleic acid comprising SEQ ID NO:11, SEQ ID NO:90, or SEQ ID NO:99.

In another embodiment, a composition of the invention comprises a mouse Hrt protein-human interacting partner protein complex wherein the human interacting partner protein comprises a molecule selected from the group consisting of sphingolipid activator protein, beta-synuclein, C11 protein, vesicle-associated membrane protein 2, SEQ ID NO:25, aldolase A, CGI106 protein, hypothalamus protein HSMNP1, alpha enolase, SEQ ID NO:35, POM-ZP3, SEQ ID NO:39, quinone oxidoreductase, SEQ ID NO:43, pumilio 1, VPS41, and KIAA0614 protein. In a further embodiment, the invention provides a composition comprising a mouse Hrt protein-human interacting partner nucleic acid complex wherein the human interacting partner nucleic acid comprises SEQ ID NO:95.

A composition comprising a mouse Hrt protein-human interacting partner protein complex where the human interacting partner protein comprises a molecule selected from the group consisting of SEQ ID NO:52, SEQ ID NO:54; SEQ ID NO:58, splicing factor CC1.4, SEQ ID NO:65, SEQ ID NO:67, ubiquitin, beta-mannosidase, SEQ ID NO:85, and SEQ ID NO:87 is a further embodiment of the invention. In a further embodiment, the human interacting partner protein is encoded by a nucleic acid comprising SEQ ID NO:50, SEQ ID NO:55, SEQ ID NO:56, SEQ ID NO:61, SEQ ID NO:62; SEQ ID NO:63, SEQ ID NO:68, SEQ ID NO:69, SEQ ID NO:72, SEQ ID NO:74, SEQ ID NO:76, SEQ ID NO:77, SEQ ID NO:78, SEQ ID NO:82, SEQ ID NO:88, SEQ ID NO: 89, or SEQ ID NO:91.

A further embodiment of the invention is a composition comprising a mouse Hrt protein-human interacting partner nucleic acid complex wherein the human interacting partner nucleic acid comprises SEQ ID NO:92, SEQ ID NO:94, SEQ ID NO:96, or SEQ ID NO:98.

A method of assaying a test compound for agonist or antagonist activity for a composition set forth herein comprises a) measuring a level of interaction between mouse Hrt protein and the human interacting partner in the absence of the test compound; b) measuring a level of interaction between mouse Hr protein and the human interacting partner in the presence of the test compound; wherein when the level measured in step b) is greater than the level in step a), the test compound has agonist activity, and wherein when the level measured in step b) is less than the level in step a), the test compound has antagonist activity.

A further embodiment of the invention is a method of inhibiting hair growth on a surface in a subject in need thereof, comprising applying to the surface a growth-inhibiting amount of a compound having antagonist activity for a composition herein for a time sufficient to inhibit hair growth on the surface.

Another embodiment of the invention is a method of increasing hair growth on a surface in a subject in need thereof, comprising applying to the surface a growth-increasing amount of a compound having agonist activity for a composition herein for a time sufficient to increase hair growth on the surface. Types of alopecias that may be treated by agents that enhance hairless protein-interacting partner complexes include androgenetic alopecia (male pattern baldness), alopecia areata, alopecia universalis, congenital alopecia universalis.

All documents cited are, in relevant part, incorporated herein by reference; the citation of any document is not to be construed as an admission that it is prior art with respect to the present invention.

### DETAILED DESCRIPTION OF THE INVENTION

A modified, improved yeast two-hybrid system was used to identify protein interaction partners with hairless protein (HR-IP). The yeast two-hybrid system, in general, measures the association of two fusion proteins expressed in yeast. Interaction discovered in yeast is indicative of potential interaction that would occur under physiological conditions.

By “hairless protein (HR)” is meant herein the mouse hairless protein. By “truncated hairless protein (HRt)” is meant the sequence provided as SEQ ID NO:103 which is amino acid residues 490-1182 of the C-terminal portion of mouse HR protein. Derivatives, fragments, or analogs of HR known to one of skill in the art in light of the present disclosure are considered equivalents of HR. An antagonist or agonist having activity for HRt is expected to also have activity for HR. Since mouse HR is greater than 80% identical to human HR, the interacting partners provided herein are expected to interact with human hairless protein. Antagonists or agonists of the present HRt-IP complexes are expected to have activity for human hairless protein interacting partner equivalents.

By “interacting partner (IP)” is meant a protein or nucleic acid that has sufficient binding affinity for HRt so that, when the IP and HRt are fused to separate constituents of a transcriptional regulator, the HRt-IP affinity is sufficient to allow reconstitution of the constituents of the transcriptional regulator thereby providing an activated regulator. The interaction may occur due to specific electrostatic, hydrophobic, hydrophilic, entropic or other interaction of certain portions of HRt with certain portions of IP to form a stable complex under conditions effective to promote the interaction. Interacting partners are identified by the data provided in Table 1. That is, the identity of the interacting partner was determined by homology searching using nucleotide sequence data from positive results in the yeast two-hybrid system. Derivatives, fragments, or analogs of interacting partners identified in Table 1 known to one of skill in the art in light of the present disclosure are considered equivalents of interacting partners.

By “HRt-IP complex” is meant at least one molecule of HRt associated with at least one molecule of interacting partner, under physiological conditions of ionic strength, temperature, pH and the like. The complex may be in vivo or in vitro.

In the yeast two-hybrid system, transcription of a reporter gene (e.g., gal4) is dependent upon reconstitution of a gal 4 regulator by the interaction of two proteins, each fused to “half” of the regulator. When the two proteins have sufficient binding affinity to interact, the interaction results in the reconstitution of the gal 4 regulator which then activates transcription of the reporter gene. “Half” of the regulator is a DNA binding domain (BD), and “half” of the regulator is an activation domain (AD). When a “bait” protein is fused to the binding domain, and a “prey” protein is fused to the activator domain, or vice versa, sufficient binding affinity of the “bait” and “prey” proteins allow reconstitution of BD and AD to allow the reporter gene to be activated. Such an assay therefore is a test for affinity between the “bait” and “prey” proteins. The yeast two hybrid technology has been described in, for example, U.S. Pat. No. 5,986,055, Nov. 16, 1999 to Yang, M., et al., incorporated by reference herein.

In the present invention, the “bait” protein is a C-terminal portion of hairless protein of mouse (HRt) having amino acid residues 490 to 1182 (provided as SEQ ID NO:103, the nucleic acid sequence encoding amino acids 490-1182 is provided as SEQ ID NO:102) “Structure and Expression of the Hairless Gene of Mice,” Begona, M., et al., J. Proc. Natl. Acad. Sci, USA 91:7717-7721, 1994) (GenBank accession no. Z32675).

In the present invention, the “prey” protein is a human brain cDNA library. When two complimentary mating types of yeast, one containing the BD-HRt fusion protein, and the other containing the AD-human cDNA protein, are mated, the progeny express each fusion protein. Those cDNA proteins having sufficient affinity for HRt will bind HRt and allow the BD and AD to reconstitute to activate a reporter gene. Therefore, positive results from the two-hybrid system demonstrate affinity and interaction between the mouse HRt and a human protein.

Since mouse Hr is greater than 80% identical to human HR, the present inventors would expect with reasonable certainty that the human proteins that interact with mouse HRt provided by the present invention would also interact with human HRt. Similarly, test-compounds that prove to be agonists or antagonists of a mouse HRt-human interacting partner interaction are expected by the present inventors to be agonists or antagonists of the human HRt-human interacting partner.

In addition to providing HRt-human interacting protein complexes, the assay used herein resulted in certain positive results for untranslated 3′ regions of DNA as well as to RNA. While not wanting to be bound by theory, the present inventors believe that BD-HRt may bind mitochondrial 16S rRNA, for example, and that interaction may cause transcriptional activation (for example, 16S rRNA present close to promoter by virtue of its interaction with BD-HRt may as such or through its secondary interaction with a yeast transcriptional activator may activate transcription.

By an “antagonist” of HR-IP interaction is meant an agent having inhibitory activity for the binding of the HR-IP complex. The binding may be inhibited by an effect on the interaction between HR and IP, or by an effect on HR or IP that affects the interaction between HR and IP.

By an “agonist” of HR-IP interaction is meant an agent having enhancing or stimulatory activity for the binding of the HR-IP complex. The binding may be stimulated by an effect on the interaction between HR and IP or by an effect on HR or IP that affects the interaction between HR and IP. Identification of an antagonist or an agonist is made by allowing HR and IP to interact in the presence of a test agent. A decrease or increase in HR-IP interaction relative to the interaction when the test agent is absent indicates that the test agent has an effect on the interacting pair as further described infra.

In HR-interacting partner protein compositions of the present invention, conservative amino acid substitutions, such as Glu/Asp, Val/Ile, Ser/Thr, Arg/Lys and Gln/Asn, would be considered equivalent since the chemical similarity of these pairs of amino acid residues would be expected to result in functional equivalency. Amino acid substitutions that conserve the biological function of the HR-interacting partner protein would conserve such properties as hydrophobicity, hydrophilicity, side-chain charge, or size. Functional equivalency is determined by the interaction of the equivalent pair as compared to the native pair. Included within the scope of the invention are complexes of HR-interacting protein fragments, derivatives, or analogs that are modified during or after translation, e.g., by glycosylation, acetylation, phosphorylation, amidation, fatty acylation, sulfation, derivatization by known protecting/blocking groups, proteolytic cleavage, linkage to an antibody molecule or other cellular ligand, or the like.

In HR-interacting partner nucleic acid compositions of the present invention, a molecule that is the complement of a nucleic acid molecule that hybridizes to the interacting partner nucleic acid under stringent hybridization conditions would be considered equivalent since such a molecule is expected to result in functional equivalency as an interacting partner nucleic acid. Functional equivalency is determined by the interaction of the equivalent pair as compared to the native pair. Stringency conditions for hybridization depend upon a number of factors such as the length of hybridizing molecules, salt concentration, temperature, and the presence or absence of denaturing agents, for example. High stringency conditions may include hybridization at about 42° C. and about 50% formamide, 0.1 mg/mL sheared salmon sperm DNA, 1% SDS, 2×SSC, 10% Dextran sulfate, a first wash at about 65° C., about 2×SSC, and 1% SDS, followed by a second wash at about 65° C. and about O. 1×SSC. Alternatively, high stringency conditions may include hybridization at about 42° C. and about 50% formamide, 0.1 mg/mL sheared salmon sperm DNA, 0.5% SDS, 5×SSPE, 1× Denhardt's, followed by two washes at room temperature and 2×SSC, 0.1% SDS, and two washes at between 55-60° C. and 0.2×SSC, 0.1% SDS.

HR-IP complexes may be used for preparation of polyclonal or monoclonal antibodies, antibody fragments, humanized antibodies, single chain antibodies for affinity purification, detection or other functional studies. Methods for producing antibodies are well known in the art and can be applied to HR-IP complexes in light of the present disclosure. Antibodies having binding specificity for the HR portion or the IP portion of the HR-IP complex may be removed from an anti-HR-IP preparation by adsorption with HR or with the IP. Remaining antibodies will have specificity for the complex.

Screening for Antagonists or Agonists of HR-IP Complexes

HR-IP complexes or nucleic acids encoding HR-IP complexes may be used to screen for compounds that bind to HR-IP complexes and thus, such compounds have use as agonists or antagonists of HR-IP complexes. The invention therefore provides assays to detect molecules that either enhance or inhibit interaction of HR and an interacting partner. Such molecules may be small molecules, peptides, modified peptides, aptamers, nucleic acids, or modified nucleic acids, for example. A molecule that modulates activity of HR-IP is identified by contacting a test molecule either with HR or with IP prior to contacting HR and IP together, or allowing a three way binding among HR, IP and the test molecule. Modulation of HR-IP interaction may be measured by measuring the stability of the complex, the affinity or binding of the complex, rate of formation of the complex, the amount of complex, or another function of the complex. An increase or decrease in any of these parameters relative to the parameter in the absence of the test molecule indicates that the test molecule is an agonist or an antagonist of the complex. Methods for identifying such agonists or antagonists may be carried out using the modified yeast two-hybrid assay described herein, or may be carried out in vitro, for example.

Test agents to be screened for antagonist or agonist activity may be provided as mixtures of specified compounds, or as compound libraries, peptide libraries, antisera, antisense nucleic acids, random or combinatorial libraries, chemically synthesized libraries, recombinant libraries, or in vitro translation-based libraries. Agents may be screened for activity as competitive or non-competitive inhibitors of HR-IP interaction. In particular, fragments or analogs of HR or the IP may be screened for such inhibitory activity. Agents may be screened for inhibition or enhancement of binding of HR and IP under aqueous or physiological binding conditions in which HR-IP binding occurs in the absence of the agent to be tested. Agents that lessen the binding are identified as antagonists of the complex, and agents that enhance the binding are identified as agonists of the complex.

Typical binding conditions are, for example, an aqueous solution of 10-250 mM NaCl, 5-50 mM Tris-HCl, pH 5-8, 0.5% Triton X-100. Metal chelators or divalent cations may be added to improve binding. Binding temperatures may include that of an ice bath up to 42 degrees C. The time of incubation is sufficient to allow binding equilibrium to occur. Methods known to one of skill in the art in light of the present disclosure may be used to assay for binding between HR and an interacting partner and a test agent, including use of detectable labels and quantification of complex formation.

A yeast two-hybrid assay system may be used to test an agent for antagonist or agonist activity for HR-IP binding similar to the assay system set forth by U.S. Pat. No. 5,986,055, Nov. 16, 1999, for CDK2-interacting proteins, incorporated by reference herein. The two-hybrid assay is carried out as described in the present examples, except that it is done in the presence of a test agent. An increase or decrease in reporter gene activity relative to that present when the test agent is absent indicates that the test agent has an effect on the interacting pair.

Components of such a system include a reconstitutable transcriptional regulator and a reporter gene. The reconstitutable transcriptional activator comprises a DNA binding domain (BD) and an activator domain (AD) that, when reconstituted, induces transcription of the reporter gene. The nucleotide sequence encoding HRt, fragment, derivative, or analog thereof is fused to either the DNA binding domain or the activator domain, and the nucleotide sequences encoding an interacting partner identified from the data of Table 1 is fused to the other of the BD or AD. The DNA binding domain can be any DNA binding domain, as long as it specifically recognizes a DNA sequence within a promoter active for a reporter gene. The activator domain has activity for the binding domain of the transcriptional regulator. The reporter gene is operably linked to a promoter that contains a binding site for the DNA binding domain. As for example, binding of BD-HR to AD-IP leads to reconstitution of the BD-AD transcriptional regulator that activates expression of the reporter gene. The activation of transcription of the reporter gene occurs intracellularly, e.g., in prokaryotic or eukaryotic cells, preferably in cell culture. The reporter gene encodes a detectable marker molecule that can give rise to a detectable signal, e.g., a fluorescent protein or a protein that can be readily visualized or that is recognizable by a specific antibody or an enzyme such as LacZ or α-galactosidase and is readily assayed.

Strains of yeast are chosen for selectable markers that confer ability to grow under conditions that do not support the growth of cells not expressing the selectable marker, e.g., the selectable marker is an enzyme that provides an essential nutrient and the cell in which the interaction assay occurs is deficient in the enzyme and the selection medium lacks such nutrient. The reporter gene is under the control of the native promoter that naturally contains a binding site for the DNA binding protein, or under the control of a heterologous or synthetic promoter.

Examples of transcriptional regulator proteins that have separable binding and transcriptional activation domains include, for example, the GAL4, the GCN4, and the ARD1 proteins of S. cerevisiae and the human estrogen receptor. The DNA binding domain and activation domain that are employed in the fusion proteins need not be from the same transcriptional regulator. For example, a GAL4 or a LEXA DNA binding domain is employed, or a GAL4 or herpes simplex virus VPI6 activation domain is employed. In one embodiment, the yeast transcription factor GAL4 is reconstituted by the protein-protein interaction and the host strain is mutant for GAL4. Other embodiments are known to one of skill in the art in light of the present disclosure.

To facilitate isolation of the encoded interacting partners, the fusion constructs can further contain sequences encoding affinity tags such as glutathione-S-transferase or maltosebinding protein or an epitope of an HR or HRt antibody, for affinity purification for binding to the HR-IP. Where the IP is a nucleic acid, an affinity tag may be a hybridizing nucleotide sequence complementary to the nucleic acid IP.

The host cell in which the interaction assay occurs can be any cell, prokaryotic or eukaryotic, in which transcription of the reporter gene can occur and be detected, including but not limited to mammalian (e.g., monkey, chicken, mouse, rat, human, bovine), bacteria, and insect cells, and is preferably a yeast cell. Expression constructs encoding and capable of expressing the binding domain fusion proteins, the transcriptional activation domain fusion proteins, and the reporter gene product are provided within the host cell, by mating of cells containing the expression constructs, or by cell fusion, transformation, electroporation, or microinjection, for example. The host cell used should not express an endogenous transcription factor that binds to the same DNA site as that recognized by the DNA binding domain fusion population. In addition, the host cell is mutant or otherwise lacking in an endogenous, functional form of the reporter gene used in the assay.

Various vectors and host strains for expression of the two fusion protein populations in yeast are known and can be used (see, e.g., U.S. Pat. No. 5,986,055, Nov. 16, 1999). For example, yeast strains or derivative strains made therefrom, which can be used are N105, N106, N1051, N1061, and YULH, Y190: MATa, ura3-52, his3-200, Iys2-801, ade2-101, trp1-901, leu2-3,112, gal4.DELTA., gal80.DELTA., cyh.sup.r 2, LYS2::GAL1.sub.UAS-HIS3.sub.TATA HIS3, URA3::GAL1.sub.UAS-GAL1.sub.TATA-lacZ. CG-1945: MATa, ura3-52, his3-200, lys2-801, ade2-101, trp1-901, leu2-3,112, gal4-542, gal80-538, cyh.sup.r 2, LYS2::GAL1.sub.UAS HIS3.sub.TATA HIS3, URA3::GAL1.sub.UAS17mers(×3)-CYC1.sub.TATA-lacZ. Y187: MAT.alpha., ura3-52, his3-200, ade2-101, trp1-901, leu2-3,112, gal4.DELTA., gal80.DELTA., URA3::GAL1.sub.UAS-GAL1.sub.TATA-lacZ. SFY526: MATa, ura3-52, his3-200, lys2-801, ade2-101, trp1-901, leu2-3,112, gal4-542, gal80-538, can.sup.r, URA3::GAL1-lacZ. HF7c: MATa, ura3-52, his3-200, lys2-801, ade2-101, trp1-901, leu2-3,112, gal4-542, gal80-538, LYS2::GAL1-HIS3. URA3::GAL1.sub.UAS17MERS(×3)-CYC1-lacZ. YRG-2: MATa, ura3-52, his3-200, lys2-801, ade2-101, trp1-901, leu2-3,112, gal4-542, gal80-538 LYS2::GAL1.sub.UAS-GAL1.sub.TATA-HIS3, URA3::GAL1.sub.UAS 17mers(×3)-CYC1-lacZ.

Plasmids are capable of autonomous replication in a host yeast cell and preferably are capable of being propagated in E. coli. The plasmid contains a promoter directing the transcription of the DNA binding or activation domain fusion genes, and a transcriptional termination signal. The plasmid also preferably contains a selectable marker gene, permitting selection of cells containing the plasmid. The plasmid can be single-copy or multi-copy. Single-copy yeast plasmids that have the yeast centromere may also be used to express the activation and DNA binding domain fusions. In another embodiment, the fusion constructs may be introduced directly into the yeast chromosome via homologous recombination using methods known to one of skill in the art in light of the present disclosure.

Plasmids encoding the separate fusion proteins can be introduced into a single host cell (e.g., a haploid yeast cell) containing one or more reporter genes, by co-transformation, to conduct the assay for HRt-IP interactions. Alternatively, the two fusion proteins are introduced into a single cell by mating (e.g. for yeast cells) or cell fusions (e.g., of mammalian cells). In a mating type assay, conjugation of haploid yeast cells of opposite mating type that have been transformed with a binding domain fusion expression construct and an activation domain fusion expression construct, respectively, delivers both constructs into the same diploid cell. The mating type of a yeast strain may be manipulated by transformation with the HO gene.

In one embodiment, a yeast interaction mating assay is employed, using two different types of host cells, strain-types a and alpha, of the yeast Saccharomyces cerevisiae. One set of host cells, for example the a strain cells, contains fusions of the HRt nucleotide sequences with the DNA-binding domain of a transcriptional activator, such as GAL4. The hybrid proteins expressed in this set of host cells are capable of recognizing the DNA-binding site on the reporter gene. The second set of yeast host cells, for example alpha strain calls, contains nucleotide sequences as identified by the data provided in Table 1 fused to the activation domain of a transcriptional activator. In one embodiment, the fusion protein constructs are introduced into the host cell as a set of plasmids.

Bacteriophage vectors may be used to express the DNA binding domain and/or activation domain fusion proteins. Libraries can generally be prepared faster and more easily from bacteriophage vectors than from plasmid vectors.

Reporter genes include URA3, HIS3, lacZ, MEL1, GFP, luciferase, LEU2, LYS2, ADE2, TRP1, CAN1, CYH2, GUS, CUP1, or CAT, for example. Expression of LEU2, LYS2, ADE2 and TRP1 are detected by growth in a specific defined media; GUS, lacZ, MEL1, and CAT can be monitored by well known enzyme assays; and CAN1 and CYH2 are detected by selection in the presence of canavanine and cycloheximide. The natural fluorescence of GFP is detected. In another embodiment, transcription of the reporter gene is detected by a linked replication assay known to those of skill in the art in light of the present disclosure. In another embodiment, the expression of a reporter gene is detected by immunoassay. Activity of the reporter gene lacZ is monitored by measuring a detectable signal, such as X-gal (5-bromo-4-chloro-3-indolyl-.alpha.-D-galactoside).

False positives arising from transcriptional activation by the DNA binding domain fusion proteins in the absence of a transcriptional activator domain fusion protein are prevented or reduced by negative selection.

In one embodiment of the invention, the DNA sequences encoding the pairs of interacting partners are isolated by amplification in separate respective reactions. Amplification is carried out by polymerase chain reaction known by one of skill in the art using pairs of oligonucleotide primers specific for either the DNA-binding domain hybrids or the activation domain hybrids. Other amplification methods known in the art can be used, including but not limited to ligase chain reaction, use of Q beta. replicase, or the like.

Purification of HR-IP complexes

HR-IP complexes may be isolated and purified by standard methods known in the art from natural sources or recombinant host cells expressing the complexes, including but not limited to column chromatography (e.g., ion exchange, affinity, gel exclusion, reverse-phase high pressure, fast protein liquid, and the like), differential centrifugation, differential solubility, or by other standard techniques used in purification of protein complexes or protein-nucleic acid complexes.

The amino acid sequence of an interacting protein can be deduced from the nucleic acid sequence of the chimeric gene from which it was encoded. As a result, the protein or a derivative thereof can be synthesized by standard chemical or recombinant methods known in the art.

Formulations for Agonists or Antagonists of HR-IP and Administration Thereof

The concentration of an antagonist active or an agonist active, identified by methods of the present invention, in a composition may be varied over a wide range up to a saturated solution, preferably from 0.01% to 99% by weight. The maximum amount effectively applied is limited by the rate at which the active penetrates the skin. Generally, the effective amount ranges from 100 micrograms to 3000 micrograms per square centimeter of skin. The composition may be applied once or twice a day, or even more frequently, for a period of time of 2 weeks to 6 months to achieve a perceived effect on hair growth.

The composition may be administered topically by any convenient means. Topical compositions that can be applied locally to the skin may be in a form such as a solution, an oil, cream, ointment, gel, lotion, shampoo, leave-on and rinse-out hair conditioner, milk, cleanser, moisturizer, spray, skin patch, or the like.

The composition may include a non-toxic dermatologically acceptable vehicle or carrier that is adapted to be spread on the skin. Examples of suitable vehicles are acetone, alcohols, or a cream, lotion or gel that can effectively deliver the active compound. In addition, a penetration enhancer may be added to the vehicle to further enhance the effectiveness of the formulation.

A composition containing an HR-IP antagonist should be applied to an area of the body where it is desired to inhibit hair growth. A composition containing an HR-IP agonist should be applied to an area of the body where it is desired to enhance hair growth. For example, a woman may choose to inhibit hair growth on her face, arm-pits, or legs whereas a man may choose to enhance hair growth on his face, particularly to a balding area, a beard area, i.e., the cheek, neck, upper lip, or chin. An inhibitory composition may also be applied to the legs, arms, torso, bikini line, or armpit, for example. An inhibitory composition is particularly suited for hirsutism. A man may also wish to use an HR-IP antagonist as a “shaveless” product with the purpose of reducing the frequency of shaving.

Reduction of hair growth is demonstrated when the frequency of hair removal is reduced, or the subject perceives less hair on the treated site, or when the weight of hair removed by shaving is reduced. Conversely, enhancement of hair growth is demonstrated when the subject perceives more hair on the treated site, or when the weight of hair removed by shaving is increased as compared to a control.

One of ordinary skill in the art would be able to select appropriate adjunct ingredients and amounts to formulate in the compositions described above without undue experimentation, depending on the compound selected and the intended use of the composition. Suitable adjunct ingredients in topical compositions include carriers comprising one or more ingredients selected from the group consisting of i) emollients, ii) solvents, iii) humectants, iv) thickeners, v) powders, vi) perfumes, vii) waxes, viii) preservatives, ix) surfactants, x) bases, xi) penetration enhancers and others in addition to, or instead of, the adjunct ingredients listed above.

Ingredient i) is an emollient. The amount of ingredient i) in the composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, mineral oil, butyl myristate, isostearic acid, palmitic acid, isopropyl linoleate, lauryl lactate, myristyl lactate, decyl oleate, myristyl myristate, polydimethylsiloxane, petrolatum, and combinations thereof. Preferred emollients include stearyl alcohol, polydimethylsiloxane, and petrolatum.

Ingredient ii) is a solvent. The amount of ingredient ii) in the topical composition is typically about 5 to about 95%. Suitable solvents include water, ethyl alcohol, benzyl alcohol, isopropyl alcohol, castor oil, ethylene glycol monoethyl ether, diethylene glycol monobutyl ether, diethylene glycol monoethyl ether, and combinations thereof. Preferred solvents include water and benzyl alcohol.

Ingredient iii) is a humectant. The amount of ingredient iii) in the composition is typically about 2 to about 95%. Suitable humectants include glycerol, polyoxyalkylene glycols, sodium polyaspartate, methacryloyloxyethylphosphorylcholine copolymers, sodium hyaluronate, chitosan, glycerol, sorbitol, sodium 2-pyrrolidone-5-carboxylate, soluble collagen, dibutyl phthalate, gelatin, and combinations thereof. Preferred humectants include glycerol and sodium 2-pyrrolidone-5-carboxylate.

Ingredient iv) is a thickener. The amount of ingredient iv) in the composition is typically to about 95%. Thickeners include poly(acrylic acid), poly(vinyl alcohol), hydroxyethyl cellulose, methyl cellulose, carbomer [poly(acrylic acid crosslinked with polyalkenyl polyether], acrylamide copolymer, carboxymethyl starch, and combinations thereof.

Ingredient v) is a powder. The amount of ingredient v) in the composition is typically 0 to about 95%. Suitable powders include chalk, talc, fullers earth, kaolin, starch, gums, colloidal silicon dioxide, sodium polyacrylate, tetraalkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

Ingredient vi) is a perfume, as described above. The amount of ingredient vi) in the topical composition is typically about 0.001 to about 0.5%, preferably about 0.001 to about 0.1%.

Ingredient vii) is a wax. Waxes useful in this invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point between 40 and 100° C. The amount of ingredient vii) in the composition is typically about 1 to about 99%.

Ingredient viii) is a preservative such as phenol, alkyl esters of parahydroxybenzoic acid, benzoic acid and the salts thereof, boric acid and the salts thereof, sorbic acid and the salts thereof, chorbutanol, benzalkonium chloride, cetylpyridinium chloride, and parabens such as methyl paraben, ethyl paraben, and propyl paraben. Particularly preferred are the salts of benzoic acid, cetylpyridinium chloride, methyl paraben, and sodium benzoate. The amount of ingredient viii) in the composition is typically about 1 to about 99%.

Ingredient ix) is a surfactant. Suitable surfactants include lecithin, polysorbate 80, sodium lauryl sulfate, polyoxyethylene sorbitan fatty acid esters, polyoxyethylene monoalkyl ethers, sucrose monoesters, lanolin esters, lanolin ethers, and combinations thereof. Suitable surfactants are known in the art and commercially available, e.g., the TWEENS® from Atlas Powder Company of Wilmington, Del. and PEMULEN® TR-2 available from B.F. Goodrich Company of Charlotte, N.C. Suitable surfactants are disclosed in the C.T.F.A. Cosmetic Ingredient Handbook, pp.587-592 (1992); Remington: The Science and Practice of Pharmacy, 19th Ed., p. 239, 250, 1501 (1995); and McCutcheon's Volume 1, Emulsifiers & Detergents, North American Edition, pp. 236-239 (1994). The amount of ingredient ix) in the composition is typically about 1 to about 99%.

Ingredient x) is a base for adjusting the pH of the formulation. A suitable base to be used is an inorganic base, for example, sodium hydroxide, potassium hydroxide, an alkali metal oxide such as sodium or potassium oxide, ammonia, ammonium carbonate, K₂CO₃, Na₂CO₃, or an organic base such as an alkanolamines (as e.g. monoethanolamine). Typical levels of such bases, when present, are about 0.01% to about 2.0%.

Ingredient xi) is a penetration enhancer for enhancing the penetration of the actives into the skin or to the site of action. Examples of penetration enhancers include urea, propan-2-ol, polyoxyethylene ethers, terpenes, cis-fatty acids such as oleic acid or palmitoleic acid, acetone, laurocapram dimethyl sulfoxide, 2-pyrrolidone, oleyl alcohol, glyceryl-3-stearate, cholesterol, myristic acid isopropyl ester, or propylene glycol, for example. The amount of ingredient xi) in the composition is typically about 1 to about 99%. The composition also can be formulated to provide a reservoir within or on the surface of the skin to provide for a continual slow release of the active. The composition also may be formulated to evaporate slowly from the skin, allowing the active extra time to penetrate the skin.

### EXAMPLE 1

### Interaction Partners (IP's) of Hairless Protein

The present example provides for Hairless Protein (HR) interacting partners (HR-IP) using the yeast two-hybrid technology. HR cDNA corresponding to the C-terminal half of HR protein (aa residues 490 to 1182, hereinafter HRt) is cloned into a yeast two hybrid binding domain (BD) vector so as to express BD-HRt in yeast. Yeast cells expressing BD-HRt are mated with yeast cells pretransformed with human brain cDNA library in the activation domain (AD) vector. The resultant interacting partners provided by the present invention are listed in Table 1 and include molecules involved in cell cycle, cell differentiation, transcription, protein turnover, protein processing, RNA splicing, house keeping (metallothionein, aldolase, enolase) and mitochondrial machinery (quinone oxidoreductase, cytochrome oxidase II and 16S rRNA, for example). Thus, HR (and HRt) appears to be a well-connected, multi-functional protein.

Materials: A mouse early anagen cDNA library is constructed by personnel at Procter & Gamble using standard techniques. E. coli expression plasmid pET32a (Catalog No. 69015-3; component plasmid of Novagen's pET prokaryotic expression system) and E. coli strain BL21(DE3) competent cells (Catalog No. 69387-3) are purchased from Novagen (Madison, Wis.). Custom designed oligonucleotides are synthesized by personnel at Procter & Gamble using standard techniques or made by BD Biosciences Clontech (PaloAlto, Calif.). DNA sequence determination is performed by personnel at Procter & Gamble using standard techniques or contracted out to Seqwright Inc (Houston, Tex.). DNA sequence analyses and data base searches for homology are performed using SeqWeb version 1.2 (in conjunction with Wisconsin Package Version 10.1). Various microbiological media are purchased from BD Biosciences Clontech (PaloAlto, Calif.) and DIFCO Becton Dickinson Microbiology Systems (Sparks, Md.). Various premade media plates for cultivation of E. coli and yeast are purchased from Gibson Laboratories (Lexington, Ky.). Matchmaker two-hybrid System 3 (Catalog No. K1612-1), pre-transformed human brain Matchmaker cDNA library in pACT2 vector (Catalog No.HY4004AH, Lot No.9070550), Advantage 2 PCR kit (catalog No. K1910-1), and Matchmaker AD LD-insert screening amplimer sets (Catalog No. 9103-1), are purchased from BD Biosciences Clontech (PaloAlto, Calif.). Male mice (C3H strain) are purchased from Harlan Labs (Indianapolis, Ind.). NAIR lotion hair remover (4 minute formula) is purchased from a local Walgreens store. Dolgel Gel (5% ibuprofen) is purchased from Life Extension International Center Inc. (Coral Gables, Fla.). Restriction enzymes, T4 DNA ligase, molecular weight markers, agarose, and other routine molecular biology reagents are purchased from Life Technologies (Gaithersburg, Md.).

Cloning and characterization of mouse Hairless (HR) cDNA: Oligonucleotides are designed to PCR-amplify the desired portion of mouse HR cDNA, based on the published sequence for mouse HR “Structure and Expression of the Hairless Gene of Mice,” Begona, M., et al., J. Proc. Natl. Acad. Sci, USA 91:7717-7721, 1994) (GenBank accession no. Z32675). The oligonucleotides are also designed to contain restriction enzyme recognition sites for Eco RI, Xba I, Not I and Bam HI restriction enzymes for subsequent manipulation of the PCR product. In particular, the restriction enzyme sites for Eco RI and Not I (noted as underlined and italicized in the oligonucleotides shown below) are particularly useful for subsequent cloning and manipulation. The forward and reverse oligonucleotide primer pairs 5′-CCG GAA TTC GTC ACC CAG TGC CAA AGC TGT (SEQ ID NO:100) and 5′-CGG GAT CCT CTA GAG CGG CCG CTT ATT ATT TAG CTT CCT GTA ACG CCCC (SEQ ID NO:101) are used to PCR amplify HR cDNA corresponding to nucleotides 1845-3923 of HR from mouse early anagen cDNA library. PCR amplification is performed using the standard PCR protocol supplied with the Advantage 2 PCR kit. The PCR product is analyzed by agarose gel electrophoresis and is found to have the expected size (approximately 2 Kb). The PCR product is excised with Eco RI and Not I restriction enzymes. The restricted product is gel-purified and inserted into Eco RI and Not I sites of pET32a, such that the insert makes an in-frame fusion with upstream sequences. The resulting plasmid is designated pET32a-HRt. This plasmid is used for both nucleotide sequence confirmation as well as for protein expression in E. coli. The nucleotide sequence reveals changes at three positions (nucleotide position 2166, change from C to T causing a codon change from AGC to AGT; nucleotide position 2611, change from C to T causing a codon change from GAC to GAT; nucleotide position 2916, change from T to C causing a codon change from CCT to CCC) from the published sequence (SEQ ID NO:102). Amino acid number 1 of SEQ ID NO:103 corresponds to amino acid residue 490 of the C-terminal portion of HR protein. This truncated HR(HRt) cDNA corresponds to amino acid residues 490-1182 of the C-terminal portion of HR protein.

However, none of the above-cited changes results in an amino acid change. In essence the HRt cloned herein is predicted to code for a protein product identical to that of mouse HRt. The zinc finger region (amino acids 595-620) and three PEST domains are present in the 490-1182 amino acid portion of Hr. The PEST domains (region 522-546 having a score of 12.26, region 709-722 having a score of 7.56, and region 729-744 having a score of 21.71) are expected by the present inventors to be important in Hr function. Mouse HR is greater than 80% identical to human HR. PEST sequences are often found to serve as signals for rapid protein degradation. PEST sequences are constitutive or conditional signals. Constitutive means “as such,” while conditional means “upon modification” (such as phosphorylation, cis-trans isomerisation induced by light, ligand binding, etc.). To objectively determine whether a protein contains a PEST region, a computer program called PEST-FIND has been developed by M. Rechsteiner and S. W. Rogers (PEST Sequences and Regulation by Proteolysis, TIBS 21, July 1996). The algorithm, which is available in PC/GENE (OMIGA), defines a PEST sequence as a hydrophilic stretch of 12 or more amino acid residues containing at least one each of Pro, Glu/Asp, and Ser/Thr in any order or combination. They also have to be flanked by basic amino acid residues Lys, Ala, or His, but basic residues are disallowed within the PEST sequence. PEST-FIND produces a score ranging from about +50 to −50. By definition, a score >0 denotes a possible PEST region, but a value >+5 indicates a highly probable PEST region. The PEST regions noted herein also have numerous potential protein kinase C and casein kinase II phosphorylation sites. The PEST scores cited supra are among the best ever reported in the literature.

pET32a-HRt is also an inducible E. coli expression plasmid. Protein expression is studied using the standard protocol for E. coli expression as also provided by the supplier. SDS-PAGE (10-20%) analysis of total protein isolated from quadruplicate E. coli BL21(DE3)/pET32a-HRt clones induced for protein expression reveals a high level of expression of protein product of the expected size for a HRt fusion protein just after 2 h of induction. Control strain E. coli BL21(DE3)/pET32a does not show induction of any protein expression at the high molecular weight region as compared to the E. coli BL21(DE3)/pET32a-HRt clones. This study confirms expression of protein product from the HRt insert. The protein product is not recoverable under non-denaturing conditions.

Cloning of HRt into GAL4 binding domain (BD) yeast two-hybrid (Y2H) vector: The HRt insert from pET32a-HRt is excised as an Eco RI-Xho I fragment (there is a unique Xho I site just adjacent to HRt insert) and inserted at the Eco RI-Sal I sites of Y2H-BD vector pGBKT7 (Catalog No. K1612-B; a component of Matchmaker two-hybrid System 3 (Catalog No. K1612-1) BD Biosciences Clontech, Palo Alto, Calif.) to obtain pGBKT7-HRt in which HRt is fused in-frame to GAL 4 DNA BD (this plasmid is also designated BD-HRt).

Pretransformed human brain MATCHMAKER cDNA Library Screen for HRt interaction partners: Pretransformed human brain MATCHMAKER high-complexity GAL4 cDNA library that has already been amplified and introduced into yeast strain Y187 (MATα) is used to screen for HRt interacting proteins. The yeast mating partner strain PJ69-2A (MATa), provided with the pretransformed library, contains two nutritional reporter genes (ADE2 and HIS3) under the control of different promoters that significantly reduce the incidence of false positives. The library host strain Y187 provides a third reporter gene (lacZ) for optional quantification of signal strength. The protocol as described in Clontech user manual (PT3183-1) is used. Briefly, the library is mixed with PJ69-2A strain that has been transformed with the BD-HRt plasmid in YPD medium containing adenine hemisulfate (0.004%) and kanamycin (0.005%) and incubated for 20-24 h at 30° C. The next day, the mating mixture is plated on Leu, Trp, His TDO that selects for two-hybrid interactions, and then the positive clones are screened for growth on Leu, Trp, His, Ade QDO, which is a more stringent condition for interaction. Colonies that grew well on QDO are also screened for lacZ expression by streaking on QDO-Xgal plates. Based on a visual assessment of colonies for the intensity of blue color, the clones are classified into high (H), medium (M), low (L) or very low (VL) categories.

Mating efficiency is calculated to be ˜8% (by plating dilutions on leucine and tryptophan double drop out DDO plate). Nearly 4 million cells representing ˜300,000 mated cells are plated on histidine selection plate (leucine, tryptophan and histidine triple drop out, TDO plate). A total of 232 His+ colonies are obtained of which 126 colonies also grew in the absence of adenine (adenine, histidine, leucine and tryptophan quadruple drop out, QDO plate) and turned blue on QDO-Xgal plates. Based on the rate and intensity of blue color-16 are high blue, 43 are medium blue, 33 are low blue and rest is very low blue.

The positive clones are expected to contain an AD-IP (interacting partner) that interacts with BD-HRt. To characterize IP, the sequence of IP is determined and matched to the human genome database. For this purpose, total DNA is isolated from the positive clones and PCR amplified with MATCHMAKER AD LD (long distance)-insert screening amplimers (Catalog No.9103-1; BD Biosciences Clontech, PaloAlto, Calif.). Reaction is conducted in 50 μl volume for 35 cycles using the conditions recommended with the Advantage2 PCR kit (Catalog No. K1910-1; BD Biosciences Clontech, PaloAlto, Calif.). The PCR product is sequenced using the sequencing primer: 5′ CTATTCGATGATGAAGATACCCCACCAAACCCA (SEQ ID NO:104) (catalog No. 9103-1; BD Biosciences Clontech, PaloAlto, Calif.). DNA sequence analysis and data base search for homology are performed using SeqWeb version 1.2 (in conjunction with Wisconsin Package Version 10.1).

Several human interacting partners that interact with HRt are identified as listed in Table 1. Proteins having SEQ ID NO: 8, SEQ ID NO: 13, SEQ ID NO: 25, SEQ ID NO: 35, SEQ ID NO: 43, SEQ ID NO: 65, SEQ ID NO: 73, SEQ ID NO: 75, SEQ ID NO: 79, SEQ ID NO: 83 appear to be new proteins.

TABLE 1
Summary of BD-HRt(mouse) interaction with human brain c-DNA library in AD-Vector

| | | | | SEQ ID NO: | |
|---|---|---|---|---|---|
| GenBank | | | | | |
| Acc # | Clone Identity | # of hits | XGAL | DNA | ORF |
| E11456 | Human ubiquitous receptor UR | 3 + 1 | H/M | 1 | 2 |
| V00594 | Human metallothionein from cadmium-treated cells | 2 | H | 3 | 4 |
| AF055066 | Human MHC class I region | 1 | H | 11, 99 | |
| AL031676 | Human sequence from clone RP4-753, | 1 | H | 12 | 13 |
| | chromosome 21q | | | | |
| M86511 | Human monocyte antigen CD14 | 1 | H | 14 | 15 |
| M60255 | Human Sphingolipid activator protein | 3 | M | 16 | 17 |
| BC002902 | Human synuclein-beta | 1 | M | 18 | 19 |
| X81625 | Human C11 protein | 1 | M | 20 | 21 |
| BC002737 | Human vesicle-associated membrane protein 2 | 1 | M | 22 | 23 |
| AK026339 | Human small intestine cDNA to mRNA clone HSI10987 | 1 | M | 24 | 25 |
| M11560 | Human aldolase A | 3 | M | 26 | 27 |
| AF151864 | Human CGI-106 protein | 1 | M | 28 | 29 |
| AF220191 | Human hypothalamus protein HSMNP1 | 3 | M | 30 | 31 |
| M14328 | Human alpha enolase | 2 | M | 32 | 33 |
| BC006358 | Human hypothetical protein | 3 | M | 34 | 35 |
| U10099 | Human POM-ZP3 | 1 | M | 36 | 37 |
| AF159164 | Human ankyrin repeat containing protein ASB-2 | 1 | M | 38 | 39 |
| J02888 | Human quinone oxidoreductase(NQO2) | 1 | M | 40 | 41 |
| D43951 | Human pumilio(PUMH1) | 1 | M | 44 | 45 |
| U87309 | Human VPS41 | 1 | M | 46 | 47 |
| AB014514 | Human KIAA0614 protein | 1 | M | 48 | 49 |
| AF141348 | Human alpha-tubulin | 1 | L | 50 | |
| U14603 | Human tyrosine phosphatase type 1VA, member 2 | 1 | L | 51 | 52 |
| BC005003 | Human transcription factor Y, gamma | 1 | L | 53 | 54 |
| U02020 | Human pre-B cell colony enhancing factor (PBEF) | 1 | L | 55 | |
| AF016004 | Human neuronal membrane glycoprotein M6b1 | 1 | L | 56 | |
| U48437 | Human amyloid precursor like protein | 1 | L | 57 | 58 |
| L10911 | Human splicing factor (CC1.4) | 1 + 1 | L/VL | 59 | 60 |
| AK024500 | Human FLJ00109 protein | 1 | L | 61 | |
| E11295 | Human P31 | 1 | L | 62 | |
| U35376 | Human repressor transcription factor ZNF85 | 1 | L | 63 | |
| AL096810 | Human genomic region containing hypervariable mini | 1 | L | 64 | 65 |
| | satellites Chromosome 10[10q26.3] | | | | |
| AF261093 | Human Thy-1 glycoprotein | 1 | L | 66 | 67 |
| AK023181 | Human cDNA: FLJ21955 fis, clone FLJ13119 fis | 1 | L | 68 | |
| AB001899 | Human PACE4 (SPC4) | 1 | L | 69 | |
| AR075562 | Human ubiquitin | 1 + 1 | L/VL | 70 | 71 |
| BC009472 | Human cDNA FLJ20109 fis clone COL05067 | 1 | L | 72 | 73 |
| AK000463 | Human cDNA FLJ20456 fis clone KAT05827 | 1 + 2 | L/VL | 74 | 75 |
| AC005325 | Human Chromosome17, clone hRPK60 | 1 | L | 76 | |
| U46005 | Human MDC15 | 1 | L | 77 | |
| AK026840 | Human cDNA: FLJ23187 fis, clone: LNG11989 | 1 | L | 78 | 79 |
| U60337 | Human □-mannosidase | 1 | L | 80 | 81 |
| AL031847 | Human DNA sequence from clone RP1-120G22 on | 1 | L | 82 | 83 |
| | chromosome 1p36.21-36.33 (Unknown) | | | | |
| AB007896 | Human KIAA0436 | 1 | L | 84 | 85 |
| AY007158 | Human clone CDABP0113 | 1 | VL | 86 | 87 |
| AC007731 | Human chromosome 22q11 clone b562f10 | 1 | L | 88 | |
| G29373 | Human STS SHGC-32359 sequence tagged site | 1 | VL | 89 | |
| AL050038 | Human mRNA; cDNA DKFZp566J0124 | 1 | H | 90 | |
| X93334 | Human mitochondrial cytochrome oxidase III | 1 | VL | 91 | |

Homology in a 3′-untranslated region

| | | | | |
|---|---|---|---|---|
| M34641 | HUMFGF1A Human fibroblast growth factor (FGF) | 1 | VL | 92 |
| | receptor-1 mRNA | | | |
| AJ300461 | Human mRNA for C11ORF25 gene | 2 | VL | 94 |
| X83543 | Human APXL mRNA | 1 | M | 95 |
| X80818 | Human mRNA for metabotropic glutamate receptor type 4 | 1 | VL | 96 |

Homology to RNA

| | | | | |
|---|---|---|---|---|
| AF347015 | Human mitochondrial gene for 16S rRNA | 10 + 8 | M/L-VL | 98 |

Legend:
# of hits indicates the number of clones with similar sequence
XGAL
H, M, L and VL indicate rate and intensity of coloration of colonies on Xgal
H = Intense blue within 2-3 days
M = Medium blue in 3-5 days
L = Low blue in 5-7 days
VL = Very Low blue in 10 days

### EXAMPLE 2

### Screen for Compounds That Disrupt BD-HRt><AD-IP Interaction

BD-HRt><AD-IP in yeast strain. AH 109 was used for compound screening. AH109 (a component of the Matchmaker two-hybrid System 3 (Catalog No. K1612-1) BD Biosciences Clontech, Palo Alto, Calif.) is a desirable yeast reporter strain for Y2H interaction. It contains ADE2, HIS3, lacZ and MEL1 reporter genes, each of which uses a distinct GAL4-responsive promoter. The various Y2H recombinant plasmids are introduced into the yeast strain AH109 by using a lithium acetate-mediated yeast transformation protocol as provided by the supplier of the Y2H system (BD Biosciences Clontech, PaloAlto, Calif.). Transformants for combined AD and BD plasmids are selected based on their ability to grow in Leu and Trp double drop out medium (DDO).

Interaction between AD and BD fusion proteins (Y2H interaction) will bring the AD and BD domains of GAL 4 protein close to each other and thus reconstitute a functional GAL 4 protein. This will allow transformants to grow on Leu, Trp, His, Ade quadruple drop out medium (QDO). Growth rate on QDO, as well as a measure of MEL1 (alpha-galactosidase) or lacZ (beta-galactosidase) activities will give a more or less quantitative measure of avidity of Y2H interaction.

Growth inhibition of AH109 (BD-HRt><AD-IP) strain in Leu, Trp, His, Ade QDO, but not on Leu, Trp DDO is used as an initial screen for compounds for disruption of HRt-IP interaction. A diverse library of compounds is screened. Microtiter plates with 200 μl of QDO and DDO media seeded with freshly cultured yeast cells at 0.01-0.03 optical density at 600 nm (OD600) are treated with various test compounds. Stock solutions of test compounds at 20 mM are made in DMSO and tested at 400 μM final concentration. The plates are incubated without shaking at 25° C. OD600 is first measured after addition of compounds and recorded for the next 8-10 days. Wells with no compound added (positive control) are expected to reach saturation growth (˜0.7 within 3 days of incubation). Wells with only media and no cells inoculated are expected to remain at the initial OD600 value (˜0.03) over the entire period of the assay. Positive compounds from this assay are tested against an unrelated Y2H interaction (p53><T antigen) assay. pGBKT7-53 and pGADT7-T plasmids were used to establish an unrelated (to HRt) Y2H interaction in which p53 protein interacts with T antigen. These plasmids are part of the Matchmaker two-Hybrid system 3(catalog # K1612-1, BD Biosciences Clontech, Palo Alto, Calif.).

### EXAMPLE 3

### Screen for Compounds That Enhance BD-HRt><AD-IP Interaction

Increase in alpha-galactosidase activity of AH109 (BD-HRt><AD-IP) strain grown in Leu, Trp, His, Ade QDO in the presence of various test compounds is used to screen for agonists of BD-HRt><AD-IP interaction. A diverse library of compounds is screened. Microtiter plates with 200 μl of QDO media seeded with freshly cultured yeast cells at 0.05 optical density at 600 nm (OD600) are treated with various test compounds. Stock solutions of test compounds at 20 mM are made in DMSO and tested at 400 μM final concentration. The plates are incubated for 34 days without shaking at 25° C. Plates are briefly agitated to obtain uniform suspension of cells and OD600 measured. Cells are pelleted and an aliquot of medium is assayed for alpha-galactosidase activity by incubating for 10-60 minutes at 30 degree Centigrade with orthonitrophenyl alpha-D-galactoside substrate in a final volume of 100 μl. This substrate is hydrolyzed by the enzyme to ortho-nitro phenol (yellow colored product) and galactose. The reaction is terminated with 100 μl of 1M sodium carbonate solution when sufficient yellow color has developed. The amount of ortho-nitro phenol generated is determined by measuring OD at 420 nm. The alpha-galactosidase enzyme activity units is expressed as: 1000×OD 420/t×OD 600 (where t refers to elapsed incubation time in minutes). Positive compounds from this assay are tested against an unrelated Y2H interaction (p53><T antigen) assay to ensure that the compounds are specific to BD-HRt><AD-IP interaction.

### EXAMPLE 4

### Animal Model for Hair Growth Retardation (HGR) Activity

Male C3H mice (˜42-48 days old and weighing ˜24 g) purchased from Harlan are acclimatized for 2 days and ˜2 cm² area on their back is shaved closely with an electric hair trimmer. To the shaved area, −300 μl of Nair lotion (NEW! Quick'n Easy 4 Minute Formula) is thinly smeared with a gloved fingertip for ˜30 sec. The area is wiped with Kimwipes for ˜15 sec and immediately washed under a gentle stream of lukewarm water for ˜50 sec and patted dry with Kimwipes. Test materials are applied (5 animals/test material) daily for 17 days. The shaved area is visually observed every day for anagen conversion and hair growth. Animal backs are photographed on day 17. In this model Dolgel Gel (5% ibuprofen) has clear HGR activity (100% HGR activity), as evidenced by absence of any hair or pigmentation at day 17. Control treatment (60% ethanol, 20% propylene glycol and 20% Transcutol) shows clear hair growth (0% HGR activity) and show nearly 100% anagen conversion within 12 days. Test materials are compared to such controls to determine efficacy at HGR.

### EXAMPLE 5

### Animal Model for Hair Growth Stimulation (HGS) Activity

Male C3H mice (˜42-48 days old and weighing ˜24 g) purchased from Harlan are acclimatized for 2 days and ˜2 cm² area on their back is shaved closely with an electric hair trimmer. To the shaved area, ˜300 μl of Nair lotion (NEW! Quick'n Easy 4 Minute Formula) is thinly smeared with a gloved fingertip for ˜30 sec. The area is wiped with Kimwipes for ˜15 sec and immediately washed under a gentle stream of lukewarm water for ˜50 sec and patted dry with Kimwipes. Test materials are applied (5 animals/test material) daily for 17 days. The shaved area is visually observed every day for anagen conversion and hair growth. Animal backs are photographed on day 8 and 17. In this model Triiodo thyronine (T3) has clear HGS activity (100% HGS activity), as evidenced by early onset of pigmentation and clear increase in hair length at day 17 compared to control treatment (60% ethanol, 20% propylene glycol and 20% Transcutol).

All documents cited in the Detailed Description of the Invention are, in relevant part, incorporated herein by reference; the citation of any document is not to be construed as an admission that it is prior art with respect to the present invention. While particular embodiments of the present invention have been illustrated and d, it would be obvious to those skilled in the art that various other changes and ations can be made without departing from the spirit and scope of the invention. It is e intended to cover in the appended claims all such changes and modifications that are the scope of this invention.

What is claimed is:

1. A composition comprising a mouse Hrt protein-human interacting partner protein complex wherein the human interacting partner protein comprises a molecule selected from the group consisting of ubiquitous receptor UR, SEQ ID NO:4, MAP1A, SEQ ID NO:8, KIAA0930 protein, SEQ ID NO:13, and monocyte antigen CD14.

2. A composition comprising a mouse Hrt protein-human interacting partner protein complex wherein the human interacting partner protein is encoded by a nucleic acid comprising SEQ ID NO:11, SEQ ID NO:90, or SEQ ID NO:99.

3. A composition comprising a mouse Hrt protein-human interacting partner protein complex wherein the human interacting partner protein comprises a molecule selected from the group consisting of sphingolipid activator protein, beta-synuclein, C11 protein, vesicle-associated membrane protein 2, SEQ ID NO:25, aldolase A, CGI-106 protein, hypothalamus protein HSMNP1, alpha enolase, SEQ ID NO:35, POM-ZP3, SEQ ID NO:39, quinone oxidoreductase, SEQ ID NO:43, pumilio 1, VPS41, and KIAA0614 protein.

4. A composition comprising a mouse Hrt protein-human interacting partner nucleic acid complex wherein the human interacting partner nucleic acid comprises SEQ ID NO:95.

5. A composition comprising a mouse Hrt protein-human interacting partner protein complex where the human interacting partner protein comprises a molecule selected from the group consisting of SEQ ID NO:52, SEQ ID NO:54, SEQ ID NO:58, splicing factor CC1.4, SEQ ID NO:65, SEQ ID NO:67, ubiquitin, beta-mannosidase, SEQ ID NO:85, and SEQ ID NO:87.

6. A composition comprising a mouse Hrt protein-human interacting partner protein complex wherein the human interacting partner protein is encoded by a nucleic acid comprising
SEQ ID NO:50, SEQ ID NO:55, SEQ ID NO:56, SEQ ID NO:61, SEQ ID NO:62, SEQ ID NO:63,
SEQ ID NO:68, SEQ ID NO:69, SEQ ID NO:72, SEQ ID NO:74, SEQ ID NO:76, SEQ ID NO:77,
SEQ ID NO:78, SEQ ID NO:82, SEQ ID NO:88, SEQ ID NO: 89, or SEQ ID NO:91.

7. A composition comprising a mouse Hrt protein-human interacting partner nucleic acid complex wherein the human interacting partner nucleic acid comprises SEQ ID NO:92, SEQ ID NO:94, SEQ ID NO:96, or SEQ ID NO:98.

8. A method of assaying a test compound for agonist or antagonist activity for the composition of claim 1, comprising:
a) measuring a level of interaction between mouse Hrt protein and the human interacting partner protein in the absence of the test compound;
b) measuring a level of interaction between mouse Hrt protein and the human interacting partner protein in the presence of the test compound;
wherein when the level measured in step b) is greater than the level in step a), the test compound has agonist activity, and wherein when the level measured in step b) is less than the level in step a), the test compound has antagonist activity.

9. A method of assaying a test compound for agonist or antagonist activity for the composition of claim 2, comprising:
a) measuring a level of interaction between mouse Hrt protein and the human interacting partner protein in the absence of the test compound;
b) measuring a level of interaction between mouse Hrt protein and the human interacting partner protein in the presence of the test compound;
wherein when the level measured in step b) is greater than the level in step a), the test compound has agonist activity, and wherein when the level measured in step b) is less than the level in step a), the test compound has antagonist activity.

10. A method of assaying a test compound for agonist or antagonist activity for the composition of claim 3, comprising:
a) measuring a level of interaction between mouse Hrt protein and the human interacting partner protein in the absence of the test compound;
b) measuring a level of interaction between mouse Hrt protein and the human interacting partner protein in the presence of the test compound;
wherein when the level measured in step b) is greater than the level in step a), the test compound has agonist activity, and wherein when the level measured in step b) is less than the level in step a), the test compound has antagonist activity.

11. A method of assaying a test compound for agonist or antagonist activity for the composition of claim 4, comprising:
a) measuring a level of interaction between mouse Hrt protein and the human interacting partner nucleic acid in the absence of the test compound;
b) measuring a level of interaction between mouse Hrt protein and the human interacting partner nucleic acid in the presence of the test compound;
wherein when the level measured in step b) is greater than the level in step a), the test compound has agonist activity, and wherein when the level measured in step b) is less than the level in step a), the test compound has antagonist activity.

12. A method of assaying a test compound for agonist or antagonist activity for the composition of claim 5, comprising:
a) measuring a level of interaction between mouse Hrt protein and the human interacting partner protein in the absence of the test compound;
b) measuring a level of interaction between mouse Hrt protein and the human interacting partner protein in the presence of the test compound;
wherein when the level measured in step b) is greater than the level in step a), the test compound has agonist activity, and wherein when the level measured in step b) is less than the level in step a), the test compound has antagonist activity.

13. A method of assaying a test compound for agonist or antagonist activity for the composition of claim 6, comprising:
a) measuring a level of interaction between mouse Hrt protein and the human interacting partner protein in the absence of the test compound;
b) measuring a level of interaction between mouse Hrt protein and the human interacting partner protein in the presence of the test compound;
wherein when the level measured in step b) is greater than the level in step a), the test compound has agonist activity, and wherein when the level measured in step b) is less than the level in step a), the test compound has antagonist activity.

14. A method of assaying a test compound for agonist or antagonist activity for the composition of claim 7, comprising:
a) measuring a level of interaction between mouse Hrt protein and the human interacting partner nucleic acid in the absence of the test compound;
b) measuring a level of interaction between mouse Hrt protein and the human interacting partner nucleic acid in the presence of the test compound;
wherein when the level measured in step b) is greater than the level in step a), the test compound has agonist activity, and wherein when the level measured in step b) is less than the level in step a), the test compound has antagonist activity.

15. A method of inhibiting hair growth on a surface in a subject in need thereof, comprising applying to the surface a growth-inhibiting amount of a compound having antagonist activity for the composition of claim 1 for a time sufficient to inhibit hair growth on the surface.

16. A method of increasing hair growth on a surface in a subject in need thereof, comprising applying to the surface a growth-increasing amount of a compound having agonist activity for the composition of claim 1 for a time sufficient to increase hair growth on the surface.

17. A method of inhibiting hair growth on a surface in a subject in need thereof, comprising applying to the surface a growth-inhibiting amount of a compound having antagonist activity for the composition of claim 2 for a time sufficient to inhibit hair growth on the surface.

18. A method of increasing hair growth on a surface in a subject in need thereof, comprising applying to the surface a growth-increasing amount of a compound having agonist activity for the composition of claim 2 for a time sufficient to increase hair growth on the surface.
